(19) United States
(12) Patent Application Publication
(10) Pub. No.: US 2013/0237511 A1
(43) Pub. Date: Sep. 12, 2013

(54) Copper Ion Delivery Platform for Pharmaceutical Agents

(71) Applicants: Thomas J. Manning, Hahira, GA (US); Ryenne Nicole Ogburn, Valdosta, GA (US); Kaitlyn Victoria Ledwitch, Valdosta, GA (US); Dennis Ray Phillips, Nicholson, GA (US); Gregory Page Wylie, Athens, GA (US)

(72) Inventors: Thomas J. Manning, Hahira, GA (US); Ryenne Nicole Ogburn, Valdosta, GA (US); Kaitlyn Victoria Ledwitch, Valdosta, GA (US); Dennis Ray Phillips, Nicholson, GA (US); Gregory Page Wylie, Athens, GA (US)

(73) Assignees: University System of Georgia, Atlanta, GA (US); Valdosta State University, Valdosta, GA (US)

(21) Appl. No.: 13/418,235

(22) Filed: Mar. 12, 2012

(51) Int. Cl. A61K 31/555; A61P 35/00

(52) U.S. Cl. 514/187; 514/185

(57) ABSTRACT

Methods for utilizing copper ions to bind to and help transport medicinal agents that contain a nitrogen atom or atoms are disclosed. The copper ion or ions serve as a delivery platform for a known pharmaceutical agent. The copper ions may be used to impact the polarity of the medicinal agents so they perform more efficiently in a physiological environment. The copper ions may also improve the efficacy of the drug by impacting their stability.

The inventor of this patent application is not an employee of the United States Government.

### BACKGROUND OF THE INVENTION

1. Field of Invention

The present invention relates to a method to increase the efficacy of any pharmaceutical compound that contains a nitrogen atom. Binding a copper ion to a pharmaceutical agent that contains a nitrogen atom can increase its water solubility, its stability and rigidity, and block the nitrogen atom from binding unwanted targets in a physiologically environment. The controlled delivery of pharmaceutical agents to a disease site or cell is a significant research challenge and needs to consider factors such as economics, reproducibility of results, delivery agent-drug complex stability and improved efficacy.

2. Description of Related Art

In the scientific literature there exist a number of methods to deliver pharmaceutical agents by binding or encasing the drug with another structure. One method being developed to improve the delivery of drugs is aptamers. Aptamers are nucleic acid sequences which can bind specific targets. For example, the formulation of aptamer-coated particles containing paclitaxel-polylactide nanoconjugates were developed to target cancer cells.

Nanoparticles composed of various organic and inorganic compositions have been developed and are in various stages of development for the delivery of medicinal agents. The role of nanoparticles is expected to both improve and provide new delivery agents for the pharmaceutical industry for decades. Nanoparticles enter the cell through a process known as endocyctosis; a process in which the material is engulfed by the cell wall.

Taxol (paclitaxel) is a natural product extracted from the bark of a yew tree. It is one of the most utilized drugs for the treatment of different cancers including breast, ovarian, central nervous system cancer (CNS), neck maladies, etc. Taxol, a mitotic inhibitor, has been produced by its ground-breaking total synthesis and semi-synthesis. While there is an extensive volume of studies relating to taxol and other taxanes, little work exists on its binding to cations, particularly any of the transition metals. The iron-taxol complex was synthesized and tested against the National Cancer Institute's sixty cell line cancer panel. The iron-taxol complex had activity significantly lower than pure taxol. Rather than enhance the taxol efficacy, iron(III) binding to the amine containing pharmaceutical agent lowered its pharmaceutical activity. This work demonstrated that any cation binding to an amine containing pharmaceutical agent will not enhance the drugs activity. There is selectivity to the copper ion.

One of the earliest nanoparticle delivery systems tested were liposomes. These systems are essentially biological micelles, having structure forms of molecular chains that have an external component which is polar and an internal component that is nonpolar. Liposomes can generally be divided into two groups (1) multilayers where there are several molecular layers composing the internal and external components (2) Unilamellar are one layer structures. The structure of the single and multilayer composites can be altered to increase or decrease water solubility and subsequently their drug delivery efficiency.

Methods have been developed that focus on specifically delivering amine containing pharmaceutical agents that are currently on the market. A U.S. patent exists that outlines a method of using bases to increase the permeation of amine drugs across the skin (U.S. Pat. No. 6,719,997). The patent covers a wide range of amine drugs which includes a variety of compounds used to treat Alzheimer's disease, enlarged prostates, and acid reflux disease.

The human protein albumin has been demonstrated to be an effective delivery platform for taxol. Albumin has higher water solubility than taxol. The use of the albumin is often referred to as a nanoparticulate formulation despite being a naturally occurring biomolecule. It has been approved, in 2005, for applications in patients with metastatic breast cancer who have been through other treatments but failed. The albumin-taxol combination is one example of the use of nanometer sized delivery agents.

In general nanoparticles are being investigated as delivery agents for many pharmaceuticals for a number of reasons including; (1) Nanoparticle size and surface parameters can be altered to achieve different transport properties (2) Nanoparticles can be designed to allow a controlled release of the drug while being transported through the patient or released (3) Nanoparticles can be administrated using different methods, including nasal, oral, intra-ocular, parenteral, and subcutaneous (4) Nanoparticles can be functionalized by molecular ligands altering which pharmaceutical site they target (5) Nanoparticles can be magnetic in nature and be guided to a specific location using magnetic fields (6) Nanoparticle composition can vary from iron oxide nanoparticles to naturally occurring proteins. While they can be as small as two or three nanometers in diameter, nanoparticles do have a high surface area and can aggregate and precipitate.

Copper sulfate (CuSO₄) has a lethality dose (LD₅₀) of approximately 30 milligrams of the copper salt per kilogram of rat (30 mg/kg). In adult humans, it requires gram quantities of copper sulfate to be lethal. In drinking water, the suggested safe level of copper is approximately 2 parts per million or 2.0 milligram/liter. In all applications proposed here, substantially lower levels of copper are proposed and the levels that would result from a typical copper (II) cation-pharmaceutical agent complex would be on par with the copper intake in a healthy diet.

For example, binding the copper (II) ion to taxol in a 1:1 complex, means that for every one mole of taxol (853 g/mol) there would be 1 mole of copper ion (63.5 g/mol) or the mass of copper would be less than ten percent the mass of taxol. Currently, taxol is sold in different formulations but some common ones are 30 milligrams (in 5 mL); 100 milligrams (in 16.7 mL), and 300 milligrams (in 50 mL) in multidose vials. In this commercially available formulation, each milliliter of the sterile solution contains 6 milligrams of taxol (paclitaxel), 527 milligrams of Cremophor® EL (polyoxyethylated castor oil) and 50% (volume/volume) of a dehydrated alcohol. In these formulations, if copper was included, the dose would contain less than one milligram of the copper cation.

The copper (II) cation has been shown to promote angiogenic responses. These observations have led to the development of anti-copper-based, anti-angiogenic strategies for the treatment of different types of cancer. Many researchers believe that Copper is a switch that turns on the angiogenesis process in tumor cells. It has been observed that patients with many types of progressive tumors typically have very high copper levels in the tumor region. Binding an amine containing drug to a copper ion will serve to block that amine site from being sidetracked by existing copper ions, in their different physiological environments. This allows the free Copper-amine complex (i.e. Cu-taxol) to by-pass the existing copper complexes, existing in the cancerous regions, and attacks its medicinal target.

Quinine is a natural product that has been used, directly and indirectly, by cultures around the world for hundreds of years. Its first recorded use was by Indians in Peru over four hundred years ago. The native Peruvian population used the bark of the cinchona tree to treat shivering and aches associated with malaria and other maladies. Spanish explorers observed this use in their 17^{th} century explorations and brought the tree back to Europe for cultivation. Since that time, extracts of the tree have been used to treat the symptoms of malaria around the world. European explorers in Africa, Central and South America, parts of the South Pacific, etc. were routinely stopped in their various quests by the onset of malaria. During significant events in the history of the United States, such as the Civil War battles in the Deep South, digging the Panama Canal and fighting in the Pacific theater during World War II, quinines presence, or lack thereof, dramatically impacted the outcome of events and the fate of the participants. For centuries the cinchona tree remained the only viable source of quinine.

In 1944 Robert Woodward and William von Eggers Doering published the total synthesis of quinine. This synthesis was significant for two reasons; the production of quinine could be attained without the cinchona tree, whose growth was limited to specific locations. During World War II there were supply problems with quinine for U.S. troops in the South Pacific. This synthesis raised hope that the supply issues could be solved. Second, the seventeen step procedure was billed as one of the first, large scale total synthesis of any natural product. It turns out that the Woodard-Doering synthesis actually did not produce quinine but a precursor that could be converted to quinine by the Rabe-Kindler synthesis, published in 1918. The Woodard-Doering and Rabe-Kindler synthesis were refuted by Gilbert Stork but, this controversy was later resolved in favor of the original authors.

Quinine (Qualaquin) has been approved by the Food and Drug Administration in treating malaria. It has been used for treating leg cramps, which is not approved by the FDA. Ingesting excessive quinine results in severe side effects including chills and fever, irregular heartbeats, loss of hearing and/or vision, yellowed skin, stomach pain and diarrhea and significant skin rashes. Excessive intake of quinine can result in death. For malaria patients, adults can be prescribed up to 500 mg per dose, taken up to three times per day. Quinine has a poor solubility in water (approximately 0.5 g/liter) but is readily soluble in ethanol and chloroform.

Quinine, a simple alkaloid, has found little use in treating cancer but it has been evaluated as a chemosensitizer in conjunction with taxol. Using quinine with taxol can increase taxol's anti-cancer activity. Understanding a medicinal agent equilibrium reaction with cations in the body can help explain their activities and side effects. Taxol, a cancer drug with a single amine, can bind copper ions (I or II), as can quinine. If quinine, with two amines, is administered with taxol but at higher concentrations, taxol's efficiency increases.

Taxol(aq)+Cu²⁺(aq)===Cu(taxol)₁(aq)K₁ (1)

Quinine+Cu²⁺(aq)===Cu(quinine)₁(aq)K₂ (2)

Taxol's increase in medical efficiency in that study can be explained as follows. Quinine binds the available or exposed naturally occurring copper in the body allowing the cancer drug to reach its medicinal target more efficiently. Lech and Sladick found that copper levels in different organs in the body (130 bodies sampled) ranged from approximately one to three parts per million or a fifty kilogram adult would have up to 0.15 grams of copper in their body. While there will be low levels of free copper in the body, most of it is bound in macromolecules involved in some essential biological function. Taxol binding copper that is already involved in an essential physiological process can not only sidetrack the taxol from its intended medicinal target but disrupt the original physiological process inducing side effects. Quinine, which has lower toxicity than taxol against all types of cancer, may bind or tie up these copper ions, allowing the taxol to reach its pharmaceutical destination with a higher degree of efficiency. Despite the water solubility limitations of common amines such as taxol and quinine, and the extensive work conducted using large structures such as nanoparticles and proteins, nothing has been done to improve the solubility using cations.

Scientists have measured the acid-base equilibrium constants (i.e. pKₐ's) of quinine as well as three other drugs that had acidic functional groups. These values were measured at different ionic strengths (0.01 to 1) and temperatures (25 and 37° C.). For quinine's two amines, pKₐ₁ was measured to be approximately 4.2 and pKₐ₂ was 8.5. pKₐ's and electron affinities of ligands have been correlated with the stability constants of metal ligand complexes. Copper-ethylene (en) stability constants have been compared to other transition metals and are typically larger and more stable. This correlation among transition metals is known as the Irving-Williams series and indicates that copper forms the strongest complexes with amines. The stability trend that follows is:

Mn(II)<Fe(II)<Co(II)<Ni(II)<Copper(II)>Zn(II) (3)

Scientists have identified a new copper(II)-quinine complex [Cu(C₂₀H₂₃O₂N₂)(OH₂)₂]ClO₄. The solid state complex was analyzed using infrared spectroscopy, electron paramagnetic resonance (EPR) and thermal analysis. The research results suggested that both amine sites were bound by Copper(II) ions but did not investigate the solution phase structure. The published work also showed the Copper(II)-quinine complex (CuQ; Q=quinine) was octahedral, not unlike most Copper(II) complexes. Given the work did not use a definitive technique to identify the structure, such as nuclear magnetic resonance, its exact structure can only be suggested.

Past quinine work in this lab involved a field project along the Suwannee River (Florida, USA) in which quinine, minus its methoxy group, was found in a number of sediment samples. This find was correlated with U.S. Civil War history in which locals used the extracts from the bark of a dogwood tree to relieve the symptoms of malaria when quinine was not available due to a naval blockade. While quinine is a well-known natural product and copper(II) a likely candidate to be investigated as a binding partner, no definitive study in the literature exists to understand the Cu₁Q₁, Cu₁Q₂ or a quinine dimer complex structure in the solution phase, which is important for medicinal applications.

The World Health Organization lists over three hundred medicines it considers essential for the various maladies that impact the entire human population. Approximately one hundred and forty of these are nitrogen containing drugs. The copper(II) ion can be used as a delivery platform for these drugs with little added expense. Table one provides the list of the drugs, the disease they are used to treat and additional information.

TABLE 1
A list of nitrogen containing drug the World Health Organization
considers essential for the basic human health needs.

| Name | Treatment | Molecular Weight | | Empirical Formula |
|---|---|---|---|---|
| Neomycin Sulfate + | Used in combination | 614.644 | g/mol | C₂₃H₄₆N₆O × |
| Bacitracin | together as a topical | | | 2½ H₂SO₄ |
| | ointment to fight infection | | | |
| | and speed up healing of | | | |
| | wounds. Together make up | | | |
| | Neosporin. | | | |
| Isoniazid + | Isoniazid by itself was one | 137.139 | g/mol | Isoniazid: C₆H₇N₃O |
| Ethambutol | of the first anti-depressants | | | Ethambutol: C₅H₁₂NO |
| | discovered, but when used in | | | |
| | combination with | | | |
| | ethambutol it is used as a | | | |
| | first line anti-tuberculosis | | | |
| | medication and prevention. | | | |
| Abacavir | A nucleoside reverse | 286.332 | g/mol | C₁₄H₁₈N₆O |
| | transcriptase inhibitor that is | | | |
| | used to treat HIV/Aids. Its | | | |
| | trade name is Ziagen and its | | | |
| | main side effect is | | | |
| | hypersensitivity. | | | |
| Sulfadoxine + | Used in combination | 310.33 | g/mol | Sulfadoxine: C₁₂H₁₄N₄O₄S |
| Pyrimethamine | together to treat and prevent | | | Pyrimethamine: C₁₂H₁₃CIN₄ |
| | malaria. Used in treatment | | | |
| | of Toxoplasma gondii | | | |
| | infections in | | | |
| | immunocompromised | | | |
| | patients such as HIV+ | | | |
| | individuals. | | | |
| Primaquine | Used in the treatment of | 259.347 | g/mol | C₁₅H₂₁N₃O |
| | malaria and Pneumocystis | | | |
| | pneumonia. It causes | | | |
| | methemoglobinemia in all | | | |
| | patients who take it. | | | |
| Sulfadiazine | Used to treat urinary tract | 250.278 | g/mol | C₁₀H₁₀N₄O₂S |
| | infections by stopping the | | | |
| | production of folic acid in | | | |
| | bacterial cell walls. Side | | | |
| | effects include loss of | | | |
| | appetite, nausea, upset | | | |
| | stomach, and dizziness. | | | |
| Levodopa + | Used in combination to treat | 197.19 | g/mol + | Leodopa: C₉H₄NO₄ |
| Carbidopa | Parkinson's disease. The | 226.229 | g/mol | Carbidopa: C₅H₇NO₂ |
| | combination of the two | | | |
| | reduces the side effects than | | | |
| | if one is used alone. | | | |
| Rifampicin + | Used in combination as first | 822.94 | g/mol + | Pyrizinamide: C₅H₅N₃O |
| Pyrazinamide + | line defense against | 123.113 | g/mol + | |
| Isoniazide + | tuberculosis. First | 137.139 | g/mol + | |
| Ethambutol | phase/line dosaging for | 204.31 | g/mol | |
| | tuberculosis caused from | | | |
| | Mycobacterium tuberculosis. | | | |
| Trimethoprim | Treatment for prophylaxis | 290.32 | g/mol | C₁₄H₁₈N₄O₃ |
| | and urinary tract infections. | | | |
| | Also known as a | | | |
| | dihydrofolate reductase | | | |
| | inhibitor. | | | |
| Tenofovir | Used in treatment of HIV | 287.213 | g/mol | C₉H₁₄N₅O₄P |
| | and Hepatits B. Reduces | | | |
| | infection rate of both | | | |
| | viruses. | | | |
| Sulfasalazine | Used for rheumatoid | 398.394 | g/mol | C₁₈H₁₄N₄O₅S |
| | arthritis, ensethitis, and as an | | | |
| | anti-inflammatory agent in | | | |
| | inflammatory bowel disease. | | | |
| Acyclovir | Used for treatment of herpes | 225.21 | g/mol | C₈H₁₁N₅O₃ |
| | simplex virus, chicken pox, | | | |
| | and shingles. | | | |
| Propythiouracil | Treats hyperthyroidism, but | 170.233 | g/mol | C₇H₁₀N₂O₅ |
| | has a serious risk of liver | | | |
| | problems and is no longer | | | |
| | recommended as a primary | | | |
| | source of medicine. | | | |
| Chloroquine | Used in the treatment of | 319.872 | g/mol | C₁₈H₂₆ClN₃ |
| | malaria. Mildly suppresses | | | |
| | the immune system so also | | | |
| | used in some autoimmune | | | |
| | diseases. | | | |
| P-Aminosalicylic | The second antibiotic found | 153.135 | g/mol | C₇H₇NO₃ |
| Acid | to be effective in treating | | | |
| | tuberculosis. Also treats | | | |
| | inflammatory bowel disease. | | | |
| Emtricitabine | Used in treatment of HIV in | 247.248 | g/mol | C₈H₁₀FN₃O₃S |
| | adults and children. Also | | | |
| | used in treatment of hepatitis | | | |
| | B. | | | |
| Kanamycin | Used to treat many various | 484.499 | g/mol | C₁₈H₃₆N₄O₁₁ |
| | types of infections and can | | | |
| | be administered orally, | | | |
| | intravenously, or | | | |
| | intramuscularlry. | | | |
| Fluconazole | Used in the treatment and | 306.271 | g/mol | C₁₃H₁₂F₂N₆O |
| | prevention of superficial and | | | |
| | systematic fungal infections. | | | |
| Ofloxacin | A racemic mixture molecule | 361.368 | g/mol | C₁₈H₂₀FN₃O₄ |
| | which is used as a | | | |
| | chemotherapeutic antibiotic | | | |
| | of the fluoroquinine drug | | | |
| | class. | | | |
| Streptomycin | A bactericidal antibiotic | 581.574 | g/mol | C₂₁H₃₉N₇O₁₂ |
| | used as a remedy for | | | |
| | tuberculosis which is given | | | |
| | intramuscularly. | | | |
| Codeine | Used to treat moderate pain | 299.364 | g/mol | C₁₈H₂₁NO₃ |
| | and cough. Also used to | | | |
| | treat diarrhea. | | | |
| Urea | Used dermatologically to | 4.66 | debye | CH₄N₂O |
| | promote skin rehydration. | | | |
| | Treats psoriasis, xerosis, | | | |
| | eczema, keratosis and many | | | |
| | other “dry skin” diseases. | | | |
| | Urea injections are used for | | | |
| | abortions. | | | |
| Amodiaquine | Used as an anti-malarial and | 355.861 | g/mol | C₂₀H₂₂ClN₃O |
| | anti-inflammatory agent. | | | |
| | Not marketed in USA, but | | | |
| | widely used in Africa. | | | |
| Fluorouracil | Used in the treatment of | 130.077 | g/mol | C₄H₃FN₂O₂ |
| | cancer by inhibiting the | | | |
| | growth of skin cells. It will | | | |
| | harm an unborn child if used | | | |
| | as a topical agent. | | | |
| Doxycycline | Treats common | 462.46 | g/mol | C₂₂H₂₄N₂O₈ |
| | inflammation as well as | | | |
| | sinusitis, prostatits, syphilis, | | | |
| | Chlamydia, and PID. Used | | | |
| | experimentally as a matrix | | | |
| | metalloprotease inhibitor. | | | |
| Mefloquine | Used in the treatment and | 378.312 | g/mol | C₁₇H₁₆F₆N₂O |
| | prevention of mild malaria | | | |
| | infections. Can cause | | | |
| | abnormal heart rhythms. | | | |
| Miconazole | Topical treatment for fungal | 416.127 | g/mol | C₁₈H₁₄Cl₄N₂O |
| | infections; ringworm, jock- | | | |
| | itch, and athlete's foot. Kills | | | |
| | fungal cells by preventing | | | |
| | the synthesis of ergosterol. | | | |
| | Applied internally for yeast | | | |
| | infections. | | | |
| Biperiden | Used in the treatment of | 311.461 | g/mol | C₂₁H₂₉NO |
| | Parkinson's disease. | | | |
| | Relieves muscle rigidity and | | | |
| | abnormal sweating. | | | |
| Didanosine | A reverse transcriptase | 236.227 | g/mol | C₁₀H₁₂N₄O₃ |
| | inhibitor that is used as an | | | |
| | effective treatment against | | | |
| | HIV. | | | |
| Levothyroxine | Used to treat | 776.87 | g/mol | C₁₅H₁₁I₄NO₄ |
| | hypothyroidism by | | | |
| | controlling TSH. It is a | | | |
| | hormone replacement. | | | |
| Benzylpenicillin | Used in the treatment of | 334.4 | g/mol | C₁₆H₁₈N₂O₄S |
| | celluitis, bacterial | | | |
| | endocarditis, diarrhea, | | | |
| | gangrene, gonorrhea, | | | |
| | meningitis, pneumonia, | | | |
| | syphilis, septicemia, and | | | |
| | septic arthritis. | | | |
| Glibenclamide | Used in the treatment of type | 494.004 | g/mol | C₂₃H₂₈ClN₃O₅S |
| | II diabetes by stimulating | | | |
| | insulin release by inhibiting | | | |
| | ATP-sensitive potassium | | | |
| | cells in pancreatic beta cells. | | | |
| Stavudine | Inhibits HIV reverse | 224.213 | g/mol | C₁₀H₁₂N₂O₄ |
| | transcriptase by competing | | | |
| | with thymidine triphosphate. | | | |
| Lamivudine | Used to treat chronic | 229.26 | g/mol | C₈H₁₁N₃O₃S |
| | hepatitis B and HIV by | | | |
| | inhibiting reverse | | | |
| | transcriptase. | | | |
| Clotrimazole | Used to treat vaginal yeast | 334.837 | g/mol | C₂₂H₁₇ClN₂ |
| | infections, oral thrush, and | | | |
| | ringworm. | | | |
| Quinine | First effective treatment for | 324.417 | g/mol | C₂₀H₂₄N₂O₂ |
| | malaria. Very sensitive to | | | |
| | UV light. Naturally | | | |
| | occurring from the cinchoa | | | |
| | tree. | | | |
| Metformin | Oral treatment for type 2 | 129.164 | g/mol | C₄H₁₁N₅ |
| | diabetes by suppressing | | | |
| | glucose production of the | | | |
| | liver and increasing insulin | | | |
| | sensitivity. | | | |
| Oxamniquine | Used to treat worms in the | 279.3 | g/mol | C₁₄H₂₁N₃O₃ |
| | body. Causes worms to shift | | | |
| | from the mesenteric veins to | | | |
| | the liver. | | | |
| Benzathine | Used in the treatment of | 240.343 | g/mol + | Benzathine: C₁₆H₂₀N₂ |
| benzypenicillin | early or latent syphilis and | 390.4 | g/mol | Benzylpenicillin: C₁₆H₁₈N₂O₄S |
| | prevention of rheumatic | | | |
| | fever. | | | |
| Nitrofurantoin | Damages bacterial DNA and | 238.16 | g/mol | C₈H₆N₄O₅ |
| | helps in the treatment of | | | |
| | urinary tract infections. | | | |
| Efavirenz | Used in the treatment of | 315.675 | g/mol | C₁₄H₉ClF₃NO₂ |
| | HIV. Always used in | | | |
| | combination with other | | | |
| | drugs, never used alone. | | | |
| Pentamidine | Used in the prevention of | 340.42 | g/mol | C₁₉H₂₄N₄O₂ |
| | Pneumocytosis pneumonia, | | | |
| | used as a prophylactic | | | |
| | against PCP in chemo | | | |
| | patients, and treats | | | |
| | leishmaniasis and yeast | | | |
| | infections. | | | |
| Triclabendazole | Treatment for liver flukes. | 359.658 | g/mol | C₁₄H₉Cl₃N₂OS |
| | Prevents the polymerization | | | |
| | of microtubules. | | | |
| Cloxacillin | Same family of medicine as | 435.88 | g/mol | C₁₉H₁₈ClN₃O₅S |
| | penicillin and is used against | | | |
| | staphylococci that produce | | | |
| | B-lactamases. | | | |
| Chloramphenicol | Used to treat typhoid and is | 323.132 | g/mol | C₁₁H₁₂Cl₂N₂O₅ |
| | also effective against Gram- | | | |
| | positive and Gram-negative | | | |
| | bacteria. Inhibits bacterial | | | |
| | protein synthesis. | | | |
| Amikacin | An antibiotic that fights | 585.603 | g/mol | C₂₂H₄₃N₅O₁₃ |
| | against bacteria. Used to | | | |
| | treat severe, hospital- | | | |
| | acquired infections. Must be | | | |
| | administered intravenously | | | |
| | or intra muscularly. | | | |
| Ciprofloxacin | Treats infection such as | 331.346 | g/mol | C₁₇H₁₈FN₃O₃ |
| | endocarditis, gastroenteritis, | | | |
| | respiratory tract infections, | | | |
| | urinary tract infections, | | | |
| | cellulitis, anthrax and more. | | | |
| | 300 trade names. | | | |
| Ceftazidime | Used to treat bacterial | 546.58 | g/mol | C₂₂H₂₂N₆O₇S₂ |
| | infections especially those of | | | |
| | Pseudomonas aeruginosa. | | | |
| Metronidazole | Treats bacterial skin | 171.15 | g/mol | C₆H₉N₃O₃ |
| | infections of the stomach, | | | |
| | vagina, skin, joints, and | | | |
| | respiratory tract. Treats | | | |
| | dermatological conditions | | | |
| | like rosacea. | | | |
| Spectinomycin | Treats gonorrhea in patients | 332.35 | g/mol | C₁₄H₂₄N₂O₇ |
| | allergic to penicillin. | | | |
| | Interrupts bacterial protein | | | |
| | synthesis. | | | |
| Dapsone | Used for the treatment of | 248.302 | g/mol | C₁₂H₁₂N₂O₂S |
| | leprosy and Pneumocytosis | | | |
| | pneumonia. Used in multi- | | | |
| | drug therapy. | | | |
| Praziquantel | Used to treat flatworm | 312.411 | g/mol | C₁₉H₂₄N₂O₂ |
| | infections including | | | |
| | nematode, tapeworm, and | | | |
| | fluke infections. | | | |
| Ethionamide | Used as an antibiotic to treat | 166.244 | g/mol | C₈H₁₀N₂S |
| | tuberculosis. | | | |
| Cefixime | Used to treat infections | 435.452 | g/mol | C₁₆H₁₅N₅O₇S₂ |
| | caused by bacteria such as | | | |
| | bronchitis, gonorrhea, and | | | |
| | pneumonia, as well as ear, | | | |
| | lung throat, and urinary tract | | | |
| | infections. | | | |
| Ampicillin | Used to treat many bacterial | 349.41 | g/mol | C₁₆H₁₉N₃O₄S |
| | infections. | | | |
| Clindamycin | Used as a topical treatment | 424.98 | g/mol | C₁₈H₃₃ClN₂O₅S |
| | for acne and for infections | | | |
| | caused by anaerobic | | | |
| | bacteria. | | | |
| Cefazolin | Used in treatment of | 454.51 | g/mol | C₁₄H₁₄N₈O₄S₃ |
| | bacterial infections of skin, | | | |
| | lung, bone, joint, stomach, | | | |
| | blood, heart valve, and | | | |
| | urinary tract. | | | |
| Zidovudine | Used in treatment of mother- | 267.242 | g/mol | C₁₀H₁₃N₅O₄ |
| | child transmission of HIV | | | |
| | during pregnancy, labor and | | | |
| | delivery. | | | |
| Cycloserine | Used to treat tuberculosis. | 102.092 | g/mol | C₃H₆N₂O₂ |
| Nevirapine | Used in treatment of HIV-1 | 266.888 | g/mol | C₁₅H₁₄N₄O |
| | and AIDS. | | | |
| Diethylcarbamazine | Used in the treatment of | 199.293 | g/mol | C₁₀H₂₁N₃O |
| | parasites. | | | |
| Gentamicin | Synthesized by a gram- | 477.596 | g/mol | C₂₁H₄₃N₅O₇ |
| | positive bacteria and used to | | | |
| | treat many gram-negative | | | |
| | bacterial infections. | | | |
| Flucytosine | Used to treat fungal | 129.093 | g/mol | C₄H₄FN₃O |
| | infections. | | | |
| Co-amoxiclav | An antibiotic used to treat | 365.4 | g/mol + | C₁₆H₁₉N₃O₅S + |
| (combination of | against amoxicillin-resistant | 199.16 | g/mol | C₈H₉NO₅ |
| amoxicillin and | bacteria. Effective against | | | |
| clavulanic acid) | Klebsiella infections but not | | | |
| | Pseudomonas. | | | |
| Silver sulfadiazine | Used as a topical to treat | 357.14 | g/mol | C₁₀H₉AgN₄O₂S |
| | burns and prevents the | | | |
| | growth of bacteria and yeast. | | | |
| Ribavirin | Used in the treatment for | 244.206 | g/mol | C₈H₁₂N₄O₅ |
| | severed respiratory syncytial | | | |
| | virus and hepatitis C. | | | |
| Phenytoin | Suppresses abnormal brain | 252.268 | g/mol | C₁₅H₁₂N₂O₂ |
| | activity during a seizure. | | | |
| Capreomycin | Used in combination with | 668.706 | g/mol | C₂₅H₄₄N₁₄O₈ |
| | other drugs for the treatment | | | |
| | of tuberculosis. | | | |
| Procaine Penicillin | Combination of an | 236.31 | g/mol + | C₁₃H₂₀N₂O₂ + |
| | anesthetic and antibiotic. | 334.4 | g/mol | C₁₆H₁₈N₂O₄S |
| | Treats syphilis, respiratory | | | |
| | tract infections, strep throat, | | | |
| | cellulitis, and erysipelas. | | | |
| Co-Trimaoxazole | Treats upper and lower | 331.783 | g/mol | C₁₄H₁₉N₄O₃ |
| | respiratory tract infections, | | | |
| | renal urinary tract infections, | | | |
| | gastrointestinal infections, | | | |
| | and skin infections. | | | |
| Ceftriaxone | Treats community-acquired | 554.58 | g/mol | C₁₈H₁₈N₈O₇S₃ |
| | or mild to moderate health | | | |
| | care-associated pneumonia. | | | |
| | Also used to treat bacterial | | | |
| | meningitis, lyme disease, | | | |
| | typhoid fever, gonorrhea, | | | |
| | and chlamydia | | | |
| Pyrantel | Antiworm medication used | 206.31 | g/mol | C₁₁H₁₄N₂S |
| | to treat roundworm, | | | |
| | hookworm, pinworm, and | | | |
| | other worm infections. | | | |
| Mebendazole | Used to treat infestations of | 295.293 | g/mol | C₁₆H₁₃N₃O₃ |
| | worms including, pinworms, | | | |
| | roundworms, tapeworms, | | | |
| | hookworms, and whipworms. | | | |
| Levamisole | Used in the treatment of | 204.292 | g/mol | C₁₁H₁₂N₂S |
| | parasitic worm infections. Is | | | |
| | used as a “dewormer” for | | | |
| | livestock. | | | |
| Niclosamide | Used to specifically treat | 327.119 | g/mol | C₁₃H₈Cl₂N₂O₄ |
| | tapeworms and cestodes in | | | |
| | humans. | | | |
| Promethazine | Treats allergic reactions | 284.42 | g/mol | C₁₇H₂₀N₂S |
| | such as allergic rhinitis, | | | |
| | relaxes and sedates patients | | | |
| | before and after surgery or | | | |
| | during labor. | | | |
| Metoclopramide | Used to relieve heartburn | 299.8 | g/mol | C₁₄H₂₂ClN₃O₂ |
| | and speed the healing of | | | |
| | ulcers and sores in the | | | |
| | esophagus. | | | |
| Chlorpromazine | Used in the treatment of | 318.86 | g/mol | C₁₇H₁₉ClN₂S |
| | schizophrenia and other | | | |
| | psychotic disorders, as well | | | |
| | as mania in people who have | | | |
| | bipolar disorder. | | | |
| Fluphenazine | Used to treat symptoms of | 437.523 | g/mol | C₂₂H₂₈F₃N₃OS |
| | schizophrenia and psychotic | | | |
| | symptoms such as | | | |
| | hallucinations, delusions, | | | |
| | and hostility. Also treats | | | |
| | acute manic phases and | | | |
| | hostility. | | | |
| Fluoxetine | Treats major depression, | 309.33 | g/mol | C₁₇H₁₈F₃NO |
| | OCD, bulimia, and panic | | | |
| | disorder. | | | |
| Methadone | Used to manage chronic pain | 309.445 | g/mol | C₂₁H₂₇NO |
| Meglumine | A contrast dye injected into | 141.78 | g/mol | C₆H₁₅NO₅ |
| iotroxate | body before some x-ray | | | |
| | procedures. | | | |
| Phenobarbital | Commonly used to treat | 232.235 | g/mol | C₁₂H₁₂N₂O₃ |
| | neonatal seizures. It acts as | | | |
| | a central nervous system | | | |
| | depressant. Also used to | | | |
| | treat stress, anxiety, and | | | |
| | prevent withdrawal | | | |
| | symptoms of people who are | | | |
| | dependent. | | | |
| Penicillamine | Used as a form of | 149.212 | g/mol | C₅H₁₁NO₂S |
| | immunosuppressant to treat | | | |
| | rheumatoid arthritis. Also | | | |
| | used to treat Wilson's | | | |
| | disease. | | | |
| Allopurinol | Treats kidney stones and can | 136.112 | g/mol | C₅H₄N₄O |
| | lower blood pressure in mild | | | |
| | hypertension. | | | |
| Ethosuxamide | Used for treatment of | 141.168 | g/mol | C₇H₁₁NO₂ |
| | absence seizures. | | | |
| Amiloride | Treats congestive heart | 229.67 | g/mol | C₆H₈ClN₇O |
| | failure, edema associated | | | |
| | with kidney and liver | | | |
| | diseases and hypertension. | | | |
| | Also promotes the loss of | | | |
| | sodium and water from | | | |
| | body. | | | |
| Furosemide | Treats edema in people with | 340.745 | g/mol | C₁₂H₁₁ClN₂O₅S |
| | congestive heart, failure, | | | |
| | liver disease, or kidney | | | |
| | disorder. Also treats high | | | |
| | blood pressure. | | | |
| Haloperidol | Treats symptoms of | 375.9 | g/mol | C₂₁H₂₃ClFNO₂ |
| | schizophrenia, and treatment | | | |
| | of acute psychotic states and | | | |
| | delirium. Also used to | | | |
| | control motor tics in patients | | | |
| | who have Tourette's | | | |
| | disorder. | | | |
| Iohexol | Used as a contrast agent | 821.138 | g/mol | C₁₉H₂₆I₃N₃O₉ |
| | during coronary | | | |
| | angiography. | | | |
| Methotrexate | Treats rheumatoid arthritis, | 454.44 | g/mol | C₂₀H₂₂N₈O₅ |
| | certain types of cancer, and | | | |
| | treats severe psoriasis by | | | |
| | slowing growth of skin cells. | | | |
| Ranitidine | Treats ulcers and | 314.4 | g/mol | C₁₃H₂₂N₄O₃S |
| | gastroesophogeal reflux | | | |
| | disease, and helps to treat | | | |
| | Zollinger-Ellison syndrome. | | | |
| Bupropion | Is a Norepinephrine- | 239.74 | g/mol | C₁₃H₁₈ClNO |
| | dopamine reuptake inhibitor. | | | |
| | Also an antidepressant and | | | |
| | smoking cessation aid. | | | |
| Pyridoxine | Assists in the balancing of | 169.18 | g/mol | C₈H₁₁NO₃ |
| | sodium and potassium as | | | |
| | well as promoting RBC | | | |
| | production. Vitamin B6. | | | |
| Ergometrine | Facilitates the delivery of the | 325.41 | g/mol | C₁₉H₂₃N₃O₂ |
| | placenta after childbirth. | | | |
| | Causes smooth muscle tissue | | | |
| | in blood vessels to narrow, | | | |
| | reducing blood flow. | | | |
| Diazepam | Is a psychoactive drug that is | 284.7 | g/mol | C₁₆H₁₃ClN₂O |
| | used to treat anxiety, | | | |
| | insomnia, and symptoms of | | | |
| | acute alcohol withdrawal. | | | |
| Chlorhexidine | Effective on gram-positive | 505.446 | g/mol | C₂₂H₃₀Cl₂N₁₀ |
| | and gram-negative bacteria, | | | |
| | and is often used in dental | | | |
| | mouthwash to reduce dental | | | |
| | plaque and oral bacteria. | | | |
| Epinephrine | Increases heart rate, | 183.204 | g/mol | C₉H₁₃NO₃ |
| | constricts blood vessels and | | | |
| | dilates air passages. Treats | | | |
| | cardiac arrest, anaphylaxis, | | | |
| | and superficial bleeding. | | | |
| Omeprazole | Is a proton pump inhibitor | 345.4 | g/mol | C₁₇H₁₉N₃O₃S |
| | that can be given with | | | |
| | antibiotics to treat gastric | | | |
| | ulcers. Also used to treat | | | |
| | gastroesophageal reflux | | | |
| | disease. | | | |
| Sodium Calcium | Treats led poisoning, and | 292.24 | g/mol | C₁₀H₁₆N₂O₈ |
| Edetate | can take the hard metal out | | | |
| | of the blood. | | | |
| Nicotinamide | A water soluble vitamin and | 122.12 | g/mol | C₆H₆N₂O |
| | is part of the vitamin B | | | |
| | group. Treatment of patients | | | |
| | with inflammatory skin | | | |
| | conditions, and acts as a | | | |
| | chemo- and radiosensitizing | | | |
| | agent by enhancing tumor | | | |
| | blood flow. | | | |
| Methylthioninium | Treats itch, and used as an | 319.85 | g/mol | C₁₆H₁₀N₃SCl |
| Chloride | antidote for cyanide | | | |
| | poisoning and as a bacterial stain. | | | |
| Diamox | Treats conditions like | 222.245 | g/mol | C₄H₆N₄O₃S₂ |
| | glaucoma, epileptic seizures, | | | |
| | hypertension, and altitude | | | |
| | sickness. | | | |
| Ipratropium | An anticholingeric | 412.37 | g/mol | C₂₀H₃₀BrNO₃ |
| bromide | bronchodilator that blocks | | | |
| | muscarinic acetylcholine | | | |
| | receptors and opens bronchi. | | | |
| Clomipramine | Blocks serotonin, | 314.9 | g/mol | C₁₉H₂₃ClN₂ |
| | norepinephrine, and | | | |
| | dopamine transporters. Is an | | | |
| | antidepressant. | | | |
| Azathioprine | Used to prevent the rejection | 277.263 | g/mol | C₉H₇N₇O₂S |
| | of kidney transplants. Also | | | |
| | used to treat rheumatoid | | | |
| | arthritis. Weakens the | | | |
| | body's immune system. | | | |
| Naloxone | Treatment for opiate | 327.37 | g/mol | C₁₉H₂₁NO₄ |
| | overdose. Also used in the | | | |
| | treatment of congenital | | | |
| | insensitivity to pain with | | | |
| | anhidrosis. | | | |
| Carbamazepine | Treatment of seizures, | 236.269 | g/mol | C₁₅H₁₂N₂O |
| | trigeminal neuralgia, mania, | | | |
| | and bipolar I disorder. | | | |
| Thiamine | A water soluble vitamin of | 300.81 | g/mol | C₁₂ZH₁₇N₄OS |
| | the B complex. Released by | | | |
| | the action of phosphatase | | | |
| | and pyrophosphatase in the | | | |
| | upper small intestine. | | | |
| Amitriptyline | Treatment of depressive | 277.403 | g/mol | C₂₀H₂₃N |
| | disorders, anxiety disorders, | | | |
| | ADHD, migraine | | | |
| | prophylaxis, and many other | | | |
| | disorders. | | | |
| Salbutamol | Adrenergic bronchodilator | 239.311 | g/mol | C₁₃H₂₁NO₃ |
| | that opens bronchial tubes. | | | |
| | Prevents asthma, bronchitis, | | | |
| | emphysema, etc. | | | |
| Timoptol | Treats high blood pressure, | 223.678 | g/mol | C₁₃H₂₄N₄O₃S |
| | to prevent hard attacks, | | | |
| | prevents migraines, and | | | |
| | treats open-angle and | | | |
| | secondary glaucoma. | | | |
| Caffeine Citrate | Treats sever migraines. | 194.19 | g/mol | C₄H₅N₂O |
| | | | | C₃H₄O₃ |
| Tropicamide | Used to dilate the pupil and | 284.353 | g/mol | C₁₇H₂₀N₂O₂ |
| | better allows for the | | | |
| | examination of the lens, | | | |
| | vitreous humor and retina. | | | |
| Salagen | Treats Sjogren's syndrome, | 208.257 | g/mol | C₁₁H₁₆N₂O₂ |
| | chronic open-angle | | | |
| | glaucoma and acute angle- | | | |
| | closure glaucoma. | | | |
| Atropine | Lowers the parasympathetic | 289.369 | g/mol | C₁₇H₂₃NO₃ |
| | activity of muscles and | | | |
| | glands. Used to temporarily | | | |
| | paralyze the accommodation | | | |
| | reflex and to dilate the | | | |
| | pupils. | | | |
| Morphine | Opiate analgesic medication | 285.34 | g/mol | C₁₇H₁₉NO₃ |
| | used to treat severe pain. | | | |
| Amidotrizoate | Used in urography, | 613.91 | g/mol | C₁₁H₉I₃N₂O₄ |
| | venography, operative | | | |
| | cholangiography, | | | |
| | splenoportography, | | | |
| | arthrography, discography | | | |
| | and computer-assisted axial | | | |
| | tomography. | | | |
| Hydrochlorothiazide | Treats high blood pressure | 297.74 | g/mol | C₇H₈ClN₃O₄S₂ |
| | and fluid retention. Also | | | |
| | used to prevent kidney | | | |
| | stones in patients with high | | | |
| | levels of calcium in their | | | |
| | blood. | | | |
| Deferoxamine | Used to treat acute iron | 560.684 | g/mol | C₂₅H₄₈N₆O₈ |
| | poisoning, especially in | | | |
| | small children. Also used to | | | |
| | treat hemochromatosis. | | | |
| Chlorphenamine | Treats allergy symptoms | 274.788 | g/mol | C₁₆H₁₉ClN₂ |
| | such as those from hay | | | |
| | fever, hives, and runny nose. | | | |
| Riboflavin | Easily absorbed | 376.36 | g/mol | C₁₇H₂₀N₄O₆ |
| | micronutrient with a key role | | | |
| | in maintaining health in | | | |
| | humans and animals, and is | | | |
| | required for a large number | | | |
| | of cellular processes. | | | |
| DL-Methionine | Used to prevent liver | 149.21 | g/mol | C₅H₁₁NO₂S |
| | damage in acetaminophen | | | |
| | poisoning. Also used to | | | |
| | increasing acidity of urine, | | | |
| | treating liver disorders, and | | | |
| | improving wound healing. | | | |
| | Treats depression, | | | |
| | alcoholism, allergies, | | | |
| | asthma, and many other | | | |
| | disorders. | | | |
| Setraline | Antidepressant and is highly | 306.229 | g/mol | C₁₇H₁₇Cl₂N |
| | effective for the treatment of | | | |
| | panic disorder. | | | |
| Acetylcysteine | Helps loosen mucus in | 163.19 | g/mol | C₅H₉NO₃S |
| | airways. Also helps prevent | | | |
| | liver damage from | | | |
| | acetaminophen overdose. | | | |
| Nifedipine | Treats high blood pressure | 346.335 | g/mol | C₁₇H₁₈N₂O₆ |
| | and controls chest pains. | | | |
| | Increases blood supply to the | | | |
| | heart. | | | |
| Ganclovir | An antiviral medication used | 225.23 | g/mol | C₉H₁₃N₅O₄ |
| | to treat CMV. It terminates | | | |
| | elongation of viral DNA. | | | |
| Tetracaine | Local anesthetic of the ester | 264.363 | g/mol | C₁₅H₂₄N₂O₂ |
| | group. Alters the function of | | | |
| | calcium release channels. | | | |
| Ketamine | Used for the induction and | 237.725 | g/mol | C₁₃H₁₆ClNO |
| | maintenance of general | | | |
| | anesthesia usually in | | | |
| | combination with a sedative. | | | |
| Bupivacaine | Used for local anesthesia | 288.43 | g/mol | C₁₈H₁₆N₂O |
| | including infiltration, nerve | | | |
| | block, epidural, and inrathel | | | |
| | calanesthesia. | | | |
| Paracetamol | Relieves headaches and | 151.17 | g/mol | C₈H₉NO₂ |
| | minor pains. | | | |
| Lidocaine | A common local anesthetic | 234.34 | g/mol | C₁₄H₂₂N₂O |
| | and antiarrhythmic drug. | | | |
| | Helps to relieve itching, | | | |
| | burning, and pain from skin | | | |
| | inflammation. | | | |
| Ephedrine | Used as a stimulant, appetite | 165.23 | g/mol | C₁₀H₁₅NO |
| | suppressant, concentration | | | |
| | aid, decongestant, and to | | | |
| | treat hypertension associated | | | |
| | with anaesthesia. Also used | | | |
| | in the treatment of asthma, | | | |
| | bronchitis, and sea sickness. | | | |
| Thiopental | Causes drowsiness or sleep | 205.678 | g/mol | C₁₁H₁₈N₂O₂ |
| | before surgery. Depresses | | | |
| | the central nervous system | | | |
| | and helps to stop seizures. | | | |
| Tetracycline | A broad spectrum of | 444.435 | g/mol | C₂₂H₂₄N₂O₈ |
| | antibiotics that many | | | |
| | bacteria have developed | | | |
| | resistance to. Protein | | | |
| | inhibitors. | | | |
| Neostigmine | Acts as a reversible | 223.294 | g/mol | C₁₂H₁₉N₂O₂ |
| | acetylcholinsterase inhibitor. | | | |
| | Stimulates nicotinic and | | | |
| | muscarinic receptors. | | | |
| Suxamethonium | Used as a paralytic when | 290.399 | g/mol | C₁₄H₃₀N₂O₄ |
| | doing a tracheal intubation. | | | |
| Pyridostigmine | Used to treat muscle | 181.212 | g/mol | C₉H₁₃N₂O₂ |
| | weakness in people with | | | |
| | myasthenia gravis. | | | |

### BRIEF SUMMARY OF DISCLOSURE

An object of the invention is to overcome the drawbacks relating to the compromise designs of prior art devices as discussed above. Copper ions in the form of singly charged and doubly charged ions have been well studied, toxicity of the copper cations against healthy and cancerous cells are well known and reported extensively in the scientific literature.

In this invention we revealed that binding copper to any amine containing drug can potentially improve its efficacy. The copper cation binding does this by three mechanisms (1) improved water solubility (2) adds rigidity to the structure to maximize ability to lock into a specific physiological target (3) by binding the amine, the pharmaceutical agent is less likely to bind to an unwanted site causing unwanted side effects.

This binding might be by hydrogen binding to a protein to cell wall; by an ion dipole interaction to a copper containing protein that contains central copper binding sites such as Copper B centre's (Cu_{B}), Type I copper centre's (T₁Cu), Type II copper centre's (T₂Cu), Type III copper centres (T₃Cu), and Copper Z centre (Cu_{Z}).

This invention demonstrates that the copper cation does preferentially bind amines contained in the structures of the well-known medicinal agents taxol and quinine. Biological studies include the demonstration that binding taxol to iron (III) worsens the GI₅₀ values compared to uncomplexed taxol, while copper binding improves the GI₅₀ values compared to uncomplexed taxol.

### DETAILED DESCRIPTION OF THE DISCLOSURE

Quinines neutral parent ion (Q₁, C₂₀H₂₄N₂O₂) has a mass of 324.183 Dalton (Da) for the most abundant isotopic species, the copper-quinine (Cu₁C₂₀H₂₄N₂O₂; CuQ₁) has a mass of 387.113 Da, the quinine dimer (C₄₀H₄₈N₄O₄; Q₂) has a mass of 648.367 Da, and the copper (II) diquinine complex has a mass of 711.297 Da (CuQ₂; Cu₂C₄₀H₄₈N₄O₄). All masses are for the most prominent isotopic species. In mass spectral data these may appear as a (+H⁺) or m−1 (−H⁺) adducts. ⁶³Cu (69% natural abundance) and ⁶⁵Cu (31% natural abundance) are the stable isotopes of copper found in nature and provide a mass spectral pattern that is easily identified. Both Matrix Assisted Laser Desorption Ionization-Time of Flight-Mass Spectrometry (MALDI-TOF-MS) and liquid chromatography-mass spectrometry (LC-MS) were used to study the complex. With this complex, the MALDI-TOF-MS proved more useful. It revealed the presence of the parent ion (Q), CuQ₁, CuQ₂ and Q₂ complexes. Mass spectral data for the CuQ₂ complex and the experimental evidence of the quinine dimer have been published by this group. In the copper complexes the cations' unique isotopic pattern is evident in the mass spectra.

The quinine dimer (Q₂) was observed in the quinine and copper-quinine solutions. It indicates that the species can be linked without protons (m/z=648) and with amines protonated (m/z=650). The bond distances between the amines on one structure and the closest hydrogen's on the adjacent structure, coupled with the energy calculation, indicate a stable dimer structure. Table 2 and 3 provide the distances between two quinine molecules and between two quinine molecules in which the amines are protonated and linked by hydrogen bonds.

None of the mass spectrometry studies, MALDI-TOF-MS or LC-MS, indicated that chloride or water was trapped in the inner sphere of the copper-quinine complexes. Given that Copper(II) is hexavalent, this indicates that each quinine molecule in the CuQ₂ complex occupies three sites. There are six potential binding sites on each quinine molecule, the two amines (Cu—N), two oxygen (Cu—O), and two Cu-pi bonds from the ethylene and the aromatic ring.

Table 4 provides a summary of the ¹H and ¹³C Nuclear Magnetic Resonance (NMR) for the quinine and CuQ₂ complex. The shifts in position 1 (C, H atoms) indicate that the methoxy group interacts with the copper(II) ion. The lack of shifting of the entire over six member ring (#2-6) indicates its pi bonds are not involved in the binding of the copper(II) ion. The shift in positions 9 but not in position 10 indicates the amine (N #1) is involved in binding Copper(II) but not the pi bonds in the aromatic structure. The shifts in positions 11, 12 and 14 indicate the —OH and the amine (N #2) have an interaction with the copper(II). The shifts in positions #15, 17, 18, 19, and 20, which are clearly not binding sites, have shifts in their line positions due to changes in the structural changes as the natural product sticks to the Ccopper(II) ion. The shifts in carbons #9, 11, 12, 14, and 17 between the quinine and the Cu-quinine NMR experiments indicate the Copper(II) binds the two amines and the oxygen atoms. The small shifts in carbons and hydrogen numbers 1, 2, and 4 indicate the cation binding does not shift the whole structure. The shifts in carbons and hydrogen's number 19 and 20 suggest an interaction between the cation and the pi bonds. The numbering system used has been outlined in our journal articles on this topic.

In addition to the shifts in position, the spectra metal-ligand complex shows significant broadening of the spectral features which can be attributed to a rapid exchange involving the Cu—O and Cu—N bonds. This exchange, which can involve solvent or salt species, has been studied by NMR for other species such as gadolinium (III) binding DTPA. These lanthanide-aminocarboxylate complexes have been studied in the solution phase extensively because of their role as Magnetic Resonance Imaging contrast reagents. For our complexes, the following equilibrium can be suggested from the NMR and MS data;

Cu²⁺(aq)+2Q(aq) [symbol] [symbol] Cu(Q)₂⁺¹(aq)K>>1 (4)

Cu(Q)₂⁺¹(aq)+H₂O(1) [symbol] [symbol] Cu(Q)₂⁺¹(H₂O)₁(aq)K<<1 (5)

Cu(Q)₂⁺¹(aq)+Cl⁻(aq)) [symbol] [symbol] Cu(Q)₂(Cl)₁(aq)K<<1 (6)

Q(aq)+Q(aq) [symbol] [symbol] Q₂(aq)K>1 (7)

When K, the equilibrium constant, is greater than 1 it indicates there is a detectable complex. With K<1, we were not able to detect the complex. The mass spectrometer studies did not detect Cu(Q)₂⁺¹(H₂O)₁ or Cu(Q)₂(Cl)₁(aq) directly but the dynamic presence (water, chloride) in the inner sphere temporarily is suggested by the broadening of the peaks in the proton NMR experiments. Likewise, five potential binding sites on each quinine (2 Cu—N; 2 Cu—O; 1 Cu-pi/ethylene) but only three active coupled Copper(II)'s octahedral geometry indicates that three sites per quinine are in dynamic equilibrium with the cation at any given moment.

In this application, we also reveal that attaching a known medicinal agent to a copper ion can not only be used to increase water solubility and stability but also change the geometry to match other molecular complexes that have higher medicinal values. As an example, we attach two quinine molecules to a single copper cation in order to build a complex that has a similar shape and size to vinblastine and vincristine. Vinblastine and vincristine are two well-known natural products that are used in treating different types of cancers. Larger molecules can be difficult to synthesize which limits their applications in the medical community.

Table 5 shows some calculated parameters including their dipole moment, molecular volume and molecular surface area. The complexes CuQ₁; CuQ₂ and CuQ₃ were also modeled using computational chemistry software. CuQ₂ (copper(II)-(quinine)₂⁺) was found to have a number of similarities in terms of chemical and physical parameters to vincristine and vinblastine. The CuQ₂ complex was synthesized in this lab and accepted for testing at the National Cancer Institute against its sixty cancer cell line panel.

Also of note, the malarial drug hydroxychloroquine has recently been shown to impact pancreatic cancer and is entering Phase I clinical trials. The National Cancer Institute's DTP program accepted the CuQ₂ complex for testing against its 60 cancer cell line panel. The average growth rate of the cancer cells treated with the CuQ₂ complex, measured in the single dose run, increased slightly (103.70%; +/−23.38) compared to the controls (see table 6 for results). This complex performed at a similar level compared to individual tests for copper(II) sulfate as well as quinine sulfate (NSC). The results of the NCI 60 cell line panel for vinblastine and vincristine can be found on-line using the NCI-DTP COMPARE website and search engine.

The copper (II) taxol complex has also been synthesized in this lab and evaluated by the National Cancer Institute against their 60 cell line panel and modeled extensively using computational software. Tables 7, 8, and 9 provides comparative results for the National Cancer Institute results of the taxol (pure), copper(II)-taxol, and iron(III)-taxol cell line data. Table 7 is a detailed analysis between the administration of the copper(II)-taxol complex and pure taxol; table 8 is a comparison of the administration of the iron(III)-taxol complex and pure taxol; and table 9 is a comparison between the administration of the copper(II)-taxol complex and the iron(III)-taxol complex. This data clearly shows that iron-taxol complex has lower/less medicinal activity than pure taxol or the copper-taxol complex. It also demonstrates that the copper(II)-taxol complex is superior to the pure taxol molecule in terms of anti-cancer activity. The data sets were selected by using the same concentration ranges over which the drugs were applied to the cancer cell lines (10⁻⁴ to 10⁻⁸ M). In terms of medicinal activity; the CuQ₂ results show that binding copper ion to any drug doesn't make it more toxic simply because of the presence of the copper ion. Binding the iron cation to taxol and measuring a decrease in the medicinal activity shows that simply attaching any cation does not increase the drugs toxicity. Binding the copper cation to taxol and demonstrating an improvement in the medicinal activity of the well-known cancer drug shows that the copper (II) cation is a good delivery agent for medicinal products.

TABLE 2
Calculated distances between two unprotonated quinine
molecules forming a dimer in a vacuum and different
solvents. All distances are reported in Angstroms.

| Solvent | Distances Between Atoms | | | |
|---|---|---|---|---|
| vacuum | (O2,H15) = | (O2,H21) = | (H21,O2) = | |
| | 2.714 | 3.023 | 1.724 | |
| methylene | (O1,H21) = | (H12,O1) = | (H16,N2) = | |
| chloride | 2.023 | 3.183 | 3.159 | |
| ethanol | (O2,H21) = | (H21,O2) = | (H14,O2) = | (H11,N1) = |
| | 1.835 | 3.011 | 3.083 | 3.193 |
| water | (O2,H21) = | (H13,O2) = | (H21,O2) = | (H1,N1) = |
| | 1.671 | 2.616 | 3.258 | 3.140 |
| acetone | (O2,H21) = | (H21,O2) = | (H1,O1) = | (O1,H5) = |
| | 1.796 | 3.104 | 2.772 | 2.892 |

TABLE 3
Calculated distances involving the protonated amines
and hydrogen bonds in the quinine dimer. All calculated
distances are reported in Angstroms.

| Solvent | Distances Between Atoms | | | |
|---|---|---|---|---|
| vacuum | (O2,N2*) = | (O2,H11) = | (O1,H10) = | |
| | 1.747 | 2.636 | 2.661 | |
| methylene | (O2,N2*) = | (O1,H21) = | (O2,H78) = | (H20,O1) = |
| chloride | 1.876 | 2.134 | 2.978 | 3.153 |
| ethanol | (N1,H21) = | (O2,N1*) = | | |
| | 2.289 | 1.788 | | |
| water | (O2,N2*) = | (O2,H14) = | (N2,H21) = | (O1,H17) = |
| | 1.677 | 3.212 | 3.194 | 2.892 |
| acetone | (O1,N2*) = | (H5,N2) = | (O2,H17) = | |
| | 1.847 | 3.189 | 2.567 | |

TABLE 4
Experimental ¹³C and ¹H NMR data for the
quinine and the copper-quinine complexes.

| C¹³ NMR Data | | | H¹ NMR Data | | |
|---|---|---|---|---|---|
| Carbon | Quinine | Cu-Quinine | Proton | Quinine | Cu-Quinine |
| (#) | (ppm) | (ppm) | (#) | (ppm) | (ppm) |
| 1 | 57.78 | 56.03 | 1 | 3.9 | 4.06 |
| 2 | 128.64 | 127.18 | 2 | 7.35 | 7.33 |
| 3 | 131.52 | * | 3 | 7.96 | 7.97 |
| 4 | 128.68 | 127.18 | 4 | — | — |
| 5 | 128.16 | * | 5 | 7.41 | 7.45 |
| 6 | 150.75 | * | 6 | — | — |
| 7 | 142.78 | * | 7 | — | — |
| 8 | 148.184 | * | 8 | — | — |
| 9 | 144.84 | 138.06 | 9 | 8.6 | 9.11 |
| 10 | 128.66 | 127.18 | 10 | 7.65 | 7.67 |
| 11 | 72.3 | 66.82 | 11 | 4.87 | 4.87 |
| 12 | 61.12 | 59.96 | 12 | 2.22 | 2.09 |
| 13 | 28.28 | 23.53 | 13 | 1.69 | 1.29 |
| 14 | 44.18 | 43.27 | 14 | 3.01 | 3.46 |
| 15 | 41.01 | 36.57 | 15 | 1.83 | 1.91 |
| 16 | 29.25 | 26.93 | 16 | 1.49 | 1.44 |
| 17 | 56.53 | 54.06 | 17 | 2.59 | 2.78 |
| 18 | 21.68 | 18.01 | 18 | 1.35 | 0.89 |
| 19 | 123.4 | * | 19 | 5.6 | 5.71 |
| 20 | 120.09 | 115.51 | 20 | 4.85 | 5.04 |
| | | | 21 | 0.71 | 0.81 |

TABLE 5
Some calculated parameters for the Cu-quinine complexes as well as vinblastine and vincristine.

| | Cu-quinine | Cu-quinine₂ | Cu-quinine₃ | Cu-quinine₄ | vinblastine | vincristine |
|---|---|---|---|---|---|---|
| Emp. | CuC₂₀H₂₀N₂O₂ | CuC₄₀H₄₀N₄O₄ | CuC₆₀H₆₀N₆O₆ | CuC₈₀H₈₀N₈O₈ | C₄₆H₅₈N₄O₉ | C₄₆H₅₆N₄O₁₀ |
| Form. | | | | | | |
| molar | 393.01 | 716.43 | 1039.84 | 1363.26 | 810.99 | 824.97 |
| mass | | | | | | |
| surface | 397.02 | 703.72 | 975.15 | 1410.04 | 771.62 | 764.48 |
| area (Å²) | | | | | | |
| volume | 381.28 | 716.70 | 1048.89 | 1406.59 | 810.53 | 812.33 |
| (Å³) | | | | | | |
| dipole | 14.54 | 4.43 | 3.62 | 3.00 | 4.28 | 4.37 |
| moment | | | | | | |
| (Debye) | | | | | | |

TABLE 6
Results from the National Cancer Institute 60
cell line cancer panel for the Cu-Q₂ complex.

| Panel Name | Cell Panel Name | Growth Percent |
|---|---|---|
| Leukemia | CCRF-CEM | 100.1026483 |
| Leukemia | HL-60(TB) | 106.1784544 |
| Leukemia | MOLT-4 | 96.48688353 |
| Leukemia | RPMI-8226 | 105.770999 |
| Leukemia | SR | 80.31978681 |
| Non-Small Cell Lung Cancer | A549/ATCC | 88.42615546 |
| Non-Small Cell Lung Cancer | EKVX | 119.2648546 |
| Non-Small Cell Lung Cancer | HOP-62 | 132.6766986 |
| Non-Small Cell Lung Cancer | HOP-92 | 88.14007268 |
| Non-Small Cell Lung Cancer | NCI-H226 | 103.6319613 |
| Non-Small Cell Lung Cancer | NCI-H23 | 104.3440424 |
| Non-Small Cell Lung Cancer | NCI-H322M | 119.9117706 |
| Non-Small Cell Lung Cancer | NCI-H460 | 105.7854775 |
| Non-Small Cell Lung Cancer | NCI-H522 | 94.88939741 |
| Colon Cancer | COLO 205 | 91.74264468 |
| Colon Cancer | HCC-2998 | 108.9497649 |
| Colon Cancer | HCT-116 | 96.94777796 |
| Colon Cancer | HCT-15 | 98.35393057 |
| Colon Cancer | HT29 | 84.67973377 |
| Colon Cancer | KM12 | 91.03570637 |
| Colon Cancer | SW-620 | 111.0065851 |
| CNS Cancer | SF-268 | 125.3939346 |
| CNS Cancer | SF-295 | 85.69825167 |
| CNS Cancer | SF-539 | 102.385071 |
| CNS Cancer | SNB-19 | 108.7021707 |
| CNS Cancer | SNB-75 | 126.4355479 |
| CNS Cancer | U251 | 83.15718737 |
| Melanoma | LOX IMVI | 80.60818436 |
| Melanoma | MALME-3M | 105.6711816 |
| Melanoma | M14 | 101.0757053 |
| Melanoma | MDA-MB-435 | 91.46637969 |
| Melanoma | SK-MEL-2 | 95.23809524 |
| Melanoma | SK-MEL-28 | 109.1915262 |
| Melanoma | UACC-257 | 93.34409967 |
| Melanoma | UACC-62 | 84.77999268 |
| Ovarian Cancer | IGROV1 | 124.1484301 |
| Ovarian Cancer | OVCAR-3 | 119.951598 |
| Ovarian Cancer | OVCAR-4 | 110.3503826 |
| Ovarian Cancer | OVCAR-5 | 116.2155367 |
| Ovarian Cancer | OVCAR-8 | 103.7933704 |
| Ovarian Cancer | NCI/ADR-RES | 104.9770339 |
| Ovarian Cancer | SK-OV-3 | 95.06726457 |
| Renal Cancer | 786-0 | 120.528015 |
| Renal Cancer | ACHN | 100.5583965 |
| Renal Cancer | CAKI-1 | 109.9162586 |
| Renal Cancer | RXF 393 | 139.8852435 |
| Renal Cancer | SN12C | 109.4295115 |
| Renal Cancer | TK-10 | 114.9988654 |
| Renal Cancer | UO-31 | 92.23359422 |
| Prostate Cancer | DU-145 | 113.2964586 |
| Breast Cancer | MCF7 | 84.96834489 |
| Breast Cancer | MDA-MB-231/ATCC | 82.35564757 |
| Breast Cancer | HS 578T | 117.4853747 |
| Breast Cancer | BT-549 | 107.3682718 |
| Breast Cancer | T-47D | 100.0629666 |
| Breast Cancer | MDA-MB-468 | 117.8457209 |

TABLE 7
The average logGI₅₀ values (Molar) for the Copper(II)-taxol is 1.44544 times better than taxol
or (1.44 − 1.00)/(1.0) * 100 = 44% better. Fifty-one of the copper-taxol were the same
or better than pure taxol. Twenty-three of the cell lines have the same value. If both have same
GI₅₀ value, copper(II)-taxol was selected because it has higher water solubility and more likely
to perform better in animal/human trials. There are seven “missing data” because they did
not have the same set of cell lines, and three of the pure taxol cell lines outperformed copper-taxol.

| | | Copper(II)-Taxol | Taxol | Copper(II)-Taxol/ | |
|---|---|---|---|---|---|
| Panel Name | Line Name | (logGI₅₀) | (logGI₅₀) | Taxol ratio | Favored |
| Average (10^{x}) | — | −7.748 | −7.588 | 0.69183 | Cutaxol |
| Leukemia | CCRF-CEM | −8 | −8 | 1 | Cutaxol |
| Leukemia | HL-60(TB) | −8 | −8 | 1 | Cutaxol |
| Leukemia | K-562 | −8 | −7.9 | 0.79432 | Cutaxol |
| Leukemia | MOLT-4 | −8 | −7.8 | 0.6309 | Cutaxol |
| Leukemia | RPMI-8226 | −8 | −8 | 1 | Cutaxol |
| Leukemia | SR | −8 | −7.5 | 0.31622 | Cutaxol |
| Non-Small Cell Lung | A549/ATCC | −8 | −7.98 | 0.9549 | Cutaxol |
| Non-Small Cell Lung | EKVX | −8 | −6.96 | 0.0912 | Cutaxol |
| Non-Small Cell Lung | HOP-62 | −8 | −7.62 | 0.4168 | Cutaxol |
| Non-Small Cell Lung | HOP-92 | −5.29 | −7.82 | 338.84 | Taxol |
| Non-Small Cell Lung | NCI-H226 | −4.92 | −6.01 | 12.302 | Taxol |
| Non-Small Cell Lung | NCI-H23 | −8 | −7.94 | 0.8709 | Cutaxol |
| Non-Small Cell Lung | NCI-H322M | −8 | −8 | 1 | Cutaxol |
| Non-Small Cell Lung | NCI-H460 | −8 | −8 | 1 | Cutaxol |
| Non-Small Cell Lung | NCI-H522 | −8 | −8 | 1 | Cutaxol |
| Colon | COLO205 | −8 | −8 | 1 | Cutaxol |
| Colon | HCC-2998 | −8 | −7.99 | 0.9772 | Cutaxol |
| Colon | HCT-116 | −8 | −8 | 1 | Cutaxol |
| Colon | HCT-15 | −6.53 | −6.54 | 1.0232 | Taxol |
| Colon | HT29 | No Data | −8 | No Data | No Data |
| Colon | KM12 | −8 | −8 | 1 | Cutaxol |
| Colon | SW-620 | −8 | −8 | 1 | Cutaxol |
| CNS | SF-268 | −8 | −7.96 | 0.91201 | Cutaxol |
| CNS | SF-295 | −8 | −7.83 | 0.6760 | Cutaxol |
| CNS | SF-539 | −8 | −8 | 1 | Cutaxol |
| CNS | SNB-19 | −8 | −7.94 | 0.8709 | Cutaxol |
| CNS | SNB-75 | −8 | −8 | 1 | Cutaxol |
| CNS | U251 | −8 | −8 | 1 | Cutaxol |
| Melanoma | LOXIMVI | −8 | −8 | 1 | Cutaxol |
| Melanoma | MALME-3M | No Data | −6.34 | No Data | No Data |
| Melanoma | M14 | −8 | −7.99 | 0.97723 | Cutaxol |
| Melanoma | MDA-MB-435 | −8 | −7.89 | 0.77624 | Cutaxol |
| Melanoma | SK-MEL-2 | No Data | −8 | No Data | No Data |
| Melanoma | SK-MEL-28 | −8 | −6.06 | 0.01148 | Cutaxol |
| Melanoma | SK-MEL-5 | −8 | −8 | 1 | Cutaxol |
| Melanoma | UACC-257 | −8 | −6.64 | 0.04365 | Cutaxol |
| Melanoma | UACC-62 | −8 | −7.87 | 0.74131 | Cutaxol |
| Ovarian | IGROV1 | −8 | −7.74 | 0.54954 | Cutaxol |
| Ovarian | OVCAR-3 | −8 | −7.85 | 0.70794 | Cutaxol |
| Ovarian | OVCAR-4 | −8 | −5.83 | 0.0067 | Cutaxol |
| Ovarian | OVCAR-5 | −8 | −7.82 | 0.66069 | Cutaxol |
| Ovarian | OVCAR-8 | −8 | −8 | 1 | Cutaxol |
| Ovarian | NCI/ADR-RES | −5.86 | −5.72 | 0.7244 | Cutaxol |
| Ovarian | SK-OV-3 | −8 | −7.95 | 0.89125 | Cutaxol |
| Renal | 786-0 | No Data | −7.59 | No Data | No Data |
| Renal | A498 | −8 | −8 | 1 | Cutaxol |
| Renal | ACHN | −6.41 | −6.02 | 0.40738 | Cutaxol |
| Renal | CAKI-1 | −7.25 | −6.5 | 0.17782 | Cutaxol |
| Renal | RXF393 | −8 | −8 | 1 | Cutaxol |
| Renal | SN12C | −8 | −7.2 | 0.15848 | Cutaxol |
| Renal | TK-10 | −7.49 | −7.03 | 0.3467 | Cutaxol |
| Renal | UO-31 | −6.65 | −6.43 | 0.60255 | Cutaxol |
| Prostate | PC-3 | No Data | −8 | No Data | No Data |
| Prostate | DU-145 | −8 | −8 | 1 | Cutaxol |
| Breast | MCF7 | −8 | −8 | 1 | Cutaxol |
| Breast | MDA-MB-231/ATCC | −8 | −8 | 1 | Cutaxol |
| Breast | HS578T | −8 | −8 | 1 | Cutaxol |
| Breast | MDA-N | No Data | −8 | No Data | No Data |
| Breast | BT-549 | −8 | −7.9 | 0.79432 | Cutaxol |
| Breast | T-47D | No Data | −7.06 | No Data | No Data |
| Breast | MDA-MB-468 | −8 | −8 | 1 | Cutaxol |

TABLE 8
Pure taxol is 10.5 times better than the iron-taxol complex in the National
cancer Institute 60 cel line trials. In only two cases does the iron-taxol complex
have a more favorable logGI₅₀ value than pure taxol.

| | | | FERRIC- | Taxol/ | |
|---|---|---|---|---|---|
| | | Taxol | TAXOL | Fe-taxol | |
| Panel Name | Line Name | (logGI₅₀) | (logGI₅₀) | ratio | Favored |
| Average | — | −7.588 | −6.566 | 0.09506 | TAXOL |
| Panel Name | Line Name | logGI₅₀ | logGI₅₀ | 1 | TAXOL |
| Leukemia | CCRF-CEM | −8 | −6.06 | 0.01148 | TAXOL |
| Leukemia | HL-60(TB) | −8 | No Data | | No data |
| Leukemia | K-562 | −7.9 | −7.01 | 0.12882 | TAXOL |
| Leukemia | MOLT-4 | −7.8 | −4.86 | 0.00114 | TAXOL |
| Leukemia | RPMI-8226 | −8 | −7.13 | 0.13489 | TAXOL |
| Leukemia | SR | −7.5 | −6.7 | 0.15848 | TAXOL |
| Non-Small Cell Lung | A549/ATCC | −7.98 | −7.07 | 0.1230 | TAXOL |
| Non-Small Cell Lung | EKVX | −6.96 | −5.71 | 0.05623 | TAXOL |
| Non-Small Cell Lung | HOP-62 | −7.62 | −4.91 | 0.00194 | TAXOL |
| Non-Small Cell Lung | HOP-92 | −7.82 | −4.98 | 0.0014 | TAXOL |
| Non-Small Cell Lung | NCI-H226 | −6.01 | −6.12 | 1.28824 | No data |
| Non-Small Cell Lung | NCI-H23 | −7.94 | −6.79 | 0.07079 | TAXOL |
| Non-Small Cell Lung | NCI-H322M | −8 | −6.48 | 0.03019 | TAXOL |
| Non-Small Cell Lung | NCI-H460 | −8 | −7.3 | 0.19952 | TAXOL |
| Non-Small Cell Lung | NCI-H522 | −8 | −7.41 | 0.25703 | TAXOL |
| Colon | COLO205 | −8 | −7.36 | 0.22908 | TAXOL |
| Colon | HCC-2998 | −7.99 | −6.82 | 0.06760 | TAXOL |
| Colon | HCT-116 | −8 | −7.41 | 0.25703 | TAXOL |
| Colon | HCT-15 | −6.54 | −5.63 | 0.12302 | TAXOL |
| Colon | HT29 | −8 | −7.48 | 0.30199 | TAXOL |
| Colon | KM12 | −8 | −7.2 | 0.15848 | TAXOL |
| Colon | SW-620 | −8 | −7.18 | 0.15135 | TAXOL |
| CNS | SF-268 | −7.96 | −6.78 | 0.06606 | TAXOL |
| CNS | SF-295 | −7.83 | −7.08 | 0.17782 | TAXOL |
| CNS | SF-539 | −8 | −7.28 | 0.19054 | TAXOL |
| CNS | SNB-19 | −7.94 | −6.27 | 0.02137 | TAXOL |
| CNS | SNB-75 | −8 | −7.64 | 0.43651 | TAXOL |
| CNS | U251 | −8 | −7.19 | 0.15488 | TAXOL |
| Melanoma | LOXIMVI | −8 | −6.97 | 0.0933 | TAXOL |
| Melanoma | MALME-3M | −6.34 | No Data | | No data |
| Melanoma | M14 | −7.99 | −7.2 | 0.1621 | TAXOL |
| Melanoma | MDA-MB-435 | −7.89 | −7.75 | 0.7244 | TAXOL |
| Melanoma | SK-MEL-2 | −8 | −6.79 | 0.0616 | TAXOL |
| Melanoma | SK-MEL-28 | −6.06 | −4.88 | 0.06606 | TAXOL |
| Melanoma | SK-MEL-5 | −8 | −7.17 | 0.14791 | TAXOL |
| Melanoma | UACC-257 | −6.64 | −4.74 | 0.01258 | TAXOL |
| Melanoma | UACC-62 | −7.87 | −7.06 | 0.15488 | TAXOL |
| Ovarian | IGROV1 | −7.74 | −6.68 | 0.08709 | TAXOL |
| Ovarian | OVCAR-3 | −7.85 | −7.36 | 0.32359 | TAXOL |
| Ovarian | OVCAR-4 | −5.83 | −6.14 | 2.04173 | Ferric_Taxol |
| Ovarian | OVCAR-5 | −7.82 | −6.06 | 0.01737 | TAXOL |
| Ovarian | OVCAR-8 | −8 | −7.14 | 0.13803 | TAXOL |
| Ovarian | NCI/ADR-RES | −5.72 | −4.72 | 0.1 | TAXOL |
| Ovarian | SK-OV-3 | −7.95 | −6.85 | 0.07943 | TAXOL |
| Renal | 786-0 | −7.59 | −5.65 | 0.01148 | TAXOL |
| Renal | A498 | −8 | −6.23 | 0.01698 | TAXOL |
| Renal | ACHN | −6.02 | −5.53 | 0.32359 | TAXOL |
| Renal | CAKI-1 | −6.5 | −5.45 | 0.08912 | TAXOL |
| Renal | RXF393 | −8 | −7.02 | 0.10471 | TAXOL |
| Renal | SN12C | −7.2 | −6.66 | 0.2884 | TAXOL |
| Renal | TK-10 | −7.03 | −6.18 | 0.14125 | TAXOL |
| Renal | UO-31 | −6.43 | −5.31 | 0.0758 | TAXOL |
| Prostate | PC-3 | −8 | −6.66 | 0.04570 | TAXOL |
| Prostate | DU-145 | −8 | −7.22 | 0.16595 | TAXOL |
| Breast | MCF7 | −8 | −7.5 | 0.31622 | TAXOL |
| Breast | MDA-MB-231/ATCC | −8 | −6.14 | 0.01380 | TAXOL |
| Breast | HS578T | −8 | −6.85 | 0.07079 | TAXOL |
| Breast | MDA-N | −8 | No Data | | No Data |
| Breast | BT-549 | −7.9 | −6.51 | 0.04073 | TAXOL |
| Breast | T-47D | −7.06 | −7.13 | 1.17489 | Ferric_Taxol |
| Breast | MDA-MB-468 | −8 | −7.44 | 0.275422 | TAXOL |

TABLE 9
The copper-taxol complex outperforms the iron-taxol complex by 15.205 times
when comparing the GI₅₀ values measured in the NCI's 60 cell line.

| | | CU-TAXOL. | FE-TAXOL | CuTax/ | |
|---|---|---|---|---|---|
| | | CAS#1704487 | CAS#302203 | FeTax | |
| Panel Name | Line Name | (logGI₅₀) | (logGI₅₀) | ratio | Favored |
| Average | — | −7.748 | −6.566 | 0.065765784 | Cu-tax |
| Leukemia | CCRF-CEM | −8 | −6.06 | 0.011481536 | Cutax |
| Leukemia | HL-60(TB) | −8 | No Data | No Data | No data |
| Leukemia | K-562 | −8 | −7.01 | 0.102329299 | Cutax |
| Leukemia | MOLT-4 | −8 | −4.86 | 0.000724436 | Cutax |
| Leukemia | RPMI-8226 | −8 | −7.13 | 0.134896288 | Cutax |
| Leukemia | SR | −8 | −6.7 | 0.050118723 | Cutax |
| Non- Small Lung Cancer | A549/ATCC | −8 | −7.07 | 0.117489755 | Cutax |
| Non- Small Lung Cancer | EKVX | −8 | −5.71 | 0.005128614 | Cutax |
| Non- Small Lung Cancer | HOP-62 | −8 | −4.91 | 0.000812831 | Cutax |
| Non- Small Lung Cancer | HOP-92 | −5.29 | −4.98 | 0.489778819 | Cutax |
| Non- Small Lung Cancer | NCI-H226 | −4.92 | −6.12 | 15.84893192 | FeTax |
| Non- Small Lung Cancer | NCI-H23 | −8 | −6.79 | 0.0616595 | Cutax |
| Non- Small Lung Cancer | NCI-H322M | −8 | −6.48 | 0.030199517 | Cutax |
| Non- Small Lung Cancer | NCI-H460 | −8 | −7.3 | 0.199526231 | Cutax |
| Non- Small Lung Cancer | NCI-H522 | −8 | −7.41 | 0.257039578 | Cutax |
| Colon | COLO205 | −8 | −7.36 | 0.229086765 | Cutax |
| Colon | HCC-2998 | −8 | −6.82 | 0.066069345 | Cutax |
| Colon | HCT-116 | −8 | −7.41 | 0.257039578 | Cutax |
| Colon | HCT-15 | −6.53 | −5.63 | 0.125892541 | Cutax |
| Colon | HT29 | No Data | −7.48 | No data | No data |
| Colon | KM12 | −8 | −7.2 | 0.158489319 | Cutax |
| Colon | SW-620 | −8 | −7.18 | 0.151356125 | Cutax |
| CNS | SF-268 | −8 | −6.78 | 0.060255959 | Cutax |
| CNS | SF-295 | −8 | −7.08 | 0.120226443 | Cutax |
| CNS | SF-539 | −8 | −7.28 | 0.190546072 | Cutax |
| CNS | SNB-19 | −8 | −6.27 | 0.018620871 | Cutax |
| CNS | SNB-75 | −8 | −7.64 | 0.436515832 | Cutax |
| CNS | U251 | −8 | −7.19 | 0.154881662 | Cutax |
| Melanoma | LOXIMVI | −8 | −6.97 | 0.09332543 | Cutax |
| Melanoma | M14 | −8 | −7.2 | 0.158489319 | Cutax |
| Melanoma | MDA-MB-435 | −8 | −7.75 | 0.562341325 | Cutax |
| Melanoma | SK-MEL-2 | No Data | −6.79 | No data | No data |
| Melanoma | SK-MEL-28 | −8 | −4.88 | 0.000758578 | Cutax |
| Melanoma | SK-MEL-5 | −8 | −7.17 | 0.147910839 | Cutax |
| Melanoma | UACC-257 | −8 | −4.74 | 0.000549541 | Cutax |
| Melanoma | UACC-62 | −8 | −7.06 | 0.114815362 | Cutax |
| Ovarian | IGROV1 | −8 | −6.68 | 0.047863009 | Cutax |
| Ovarian | OVCAR-3 | −8 | −7.36 | 0.229086765 | Cutax |
| Ovarian | OVCAR-4 | −8 | −6.14 | 0.013803843 | Cutax |
| Ovarian | OVCAR-5 | −8 | −6.06 | 0.011481536 | Cutax |
| Ovarian | OVCAR-8 | −8 | −7.14 | 0.138038426 | Cutax |
| Ovarian | NCI/ADR-RES | −5.86 | −4.72 | 0.072443596 | Cutax |
| Ovarian | SK-OV-3 | −8 | −6.85 | 0.070794578 | Cutax |
| Renal | 786-0 | NoData | −5.65 | | no data |
| Renal | A498 | −8 | −6.23 | 0.016982437 | Cutax |
| Renal | ACHN | −6.41 | −5.53 | 0.131825674 | Cutax |
| Renal | CAKI-1 | −7.25 | −5.45 | 0.015848932 | Cutax |
| Renal | RXF393 | −8 | −7.02 | 0.104712855 | Cutax |
| Renal | SN12C | −8 | −6.66 | 0.045708819 | Cutax |
| Renal | TK-10 | −7.49 | −6.18 | 0.048977882 | Cutax |
| Renal | UO-31 | −6.65 | −5.31 | 0.045708819 | Cutax |
| Prostate | PC-3 | NoData | −6.66 | | no data |
| Prostate | DU-145 | −8 | −7.22 | 0.165958691 | Cutax |
| Breast | MCF7 | −8 | −7.5 | 0.316227766 | Cutax |
| Breast | MDA-MB-231/ATCC | −8 | −6.14 | 0.013803843 | Cutax |
| Breast | HS578T | −8 | −6.85 | 0.070794578 | Cutax |
| Breast | BT-549 | −8 | −6.51 | 0.032359366 | Cutax |
| Breast | T-47D | NoData | −7.13 | | no data |
| Breast | MDA-MB-468 | −8 | −7.44 | 0.27542287 | Cutax |

Extensive work using proton and carbon nuclear magnetic resonance, time-of-flight mass spectrometry, liquid chromatography-mass spectrometry and Fourier transform-infrared spectrometry were used to experimental characterize the copper-taxol complex. It was deemed important to establish that the copper ion actually bound the taxol molecule at the single amine, a component of the molecule that is deemed structurally less important than other molecular areas in terms of the molecules structures anti-cancer activity.

The use of NMR for the isotopes ¹H, ¹³C and ¹⁵N are essential to deduce the structure of the copper-taxol complex. Table 10 provides a summary of the experimental and literature values for the proton (¹H) and ¹³C NMR data. Some representative spectra are shown in this presentation to outline how the claim that copper (II) has an affinity for the amine is justified experimentally.

The copper-taxol complex's proton nuclear magnetic resonance spectra data demonstrated shifts in the spectra features of the pure taxol when compared to those of the copper (II)-taxol complex. This was important in establishing the copper ion did in fact bind the nitrogen atom.

A series of N¹⁵ Nuclear Magnetic Resonance spectra was measured for pure taxol and for the copper-taxol complex. The pure taxol showed two spectral peaks for the pure taxol compound indicating it had two geometries in solution. The N¹⁵ NMR spectra for the copper-taxol complex showed only a single spectral feature indicating a single geometry in solution. An analogy to this would be the well-known boat and chair geometries observed for aromatics (92 geometries), copper-taxol assumes only one of these geometries.

The N¹⁵ NMR data is important for this invention describing the utilization of the copper cation as a delivery agent for pharmaceutical agents for two reasons. First, it indicates that the copper(II) ion is in fact binding taxol at the amine. Proposing that the copper (II) ion can serve as a delivery agent to amine-containing drugs must be supported by evidence that the copper (II) binds the amine with some high rate of selectivity. Second, the cancer cell line data presented above shows that the copper (II)-taxol complexed performed better than uncomplexed taxol in the National Cancer Institute's 60 cancer cell line panel. The taxol complex has two geometries as indicated by the two spectral features in the N¹⁵ NMR. The copper (II) complex has one spectral feature indicating a single structure. Given the medicinal activity of taxol increases with the single geometry, this indicates that that geometry has more anti-cancer activity than the uncomplexed taxol. In addition to increasing water solubility, the copper (II) cation locks taxol into a single confirmation that has a preferred medicinal activity.

TABLE 10
Carbon-13 and Proton NMR data for taxol and copper (II) taxol complex are listed.

| | | | | | ¹³C Cu- | | | | | | ¹H Cu- | |
|---|---|---|---|---|---|---|---|---|---|---|---|---|
| Assignment | ¹³C Paper^{a} | | ¹³C Taxol^{b} | | Taxol^{b} | | ¹H Paper^{a} | | ¹H Taxol^{b} | | Taxol^{b} | |
| Arom 1 | o | 130.2 | o | 131.504 | o | — | o | 8.13 | o | 8.11743 | o | 8.0437 |
| | m | 128.7 | m | 120.39 | m | 129.708 | m | 7.51 | m | 7.59447 | m | 7.5139 |
| | p | 133.7 | p | 134.1 | p | 133.492 | p | 7.61 | p | 7.69707 | p | 7.60814 |
| Arom 2 | o | 127.04 | o | 127.617 | o | 127.034 | o | 7.48 | o | 7.49583 | o | 7.48069 |
| | m | 129.0 | m | 128.35 | m | 128.121 | m | 7.42 | m | 7.30198 | m | 7.42014 |
| | p | 131.9 | p | — | p | — | p | 7.35 | p | 7.30393 | p | 7.34104 |
| Arom 3 | o | 127.04 | o | 127.11 | o | | o | 7.74 | o | 7.8811 | o | 7.78978 |
| | m | 129.0 | m | 129.773 | m | 129.708 | m | 7.40 | m | — | m | 7.40012 |
| | p | 128.3 | p | 128.2 | p | 128.117 | p | 7.45 | p | — | p | 7.43626 |

| | | | | | | |
|---|---|---|---|---|---|---|
| 1′ | 172.7 | 173.085 | 172.975 | — | — | — |
| 2′ | 73.2 | — | 73.4403 | 3.61 | 3.50693 | 3.76030 |
| 3′ | 55.0 | — | 56.2854 | 5.78 | 5.66862 | — |
| 1 | 79.0 | — | — | — | — | — |
| 2 | 74.9 | — | — | 5.67 | 5.6495 | 5.58415 |
| 3 | 45.6 | — | 46.4243 | 3.79 | 3.83311 | 3.77452 |
| 4 | 81.01 | — | 80.8409 | — | — | — |
| 5 | 84.4 | — | 84.4039 | 4.94 | 4.90248 | 4.80629 |
| 6 | 35.6 | — | 35.1479 | 1.88 | 1.83061 | 1.85502 |
| 7 | 72.2 | — | — | 4.40 | 4.61585 | 4.68714 |
| 8 | 58.6 | — | 57.7998 | — | — | — |
| 9 | 203.6 | 203.8 | 203.653 | — | — | — |
| 10 | 75.5 | — | 75.340 | 6.27 | 6.47 | 6.38935 |
| 11 | 133.2 | 133.267 | — | — | — | — |
| 12 | 142.0 | 140.623 | — | — | — | — |
| 13 | 72.3 | — | — | 6.23 | 6.1745 | 6.10321 |
| 14 | 35.7 | — | 36.0481 | 2.28 | 2.27447 | 2.30377 |
| 15 | 43.2 | — | 43.1359 | — | — | — |
| 16 | 21.8 | — | 21.7734 | 1.14 | 1.17189 | 1.13918 |
| 17 | 26.9 | — | 25.447 | 1.24 | −1.80522 | 1.22317 |
| 18 | 14.8 | — | — | 1.79 | 1.67777 | 1.75248 |
| 19 | 9.5 | — | 8.9852 | 1.68 | 4.20959 | 1.60404 |
| 20 | 76.5 | — | 76.0267 | 4.19 | — | 4.13635 |
| N—H (just | — | — | — | 7.01 | — | — |
| H) | | | | | | |
| O_{ac} (Top) | 170.4 | — | — | 2.23 | 2.19146 | 2.11138 |
| O_{ac} | 171.2 | 170.035 | — | 2.38 | 2.38189 | 2.39215 |
| (Bottom) | | | | | | |
| OH (Top) | 167.02 | 166.318 | 168.839 | 2.48 | 2.48883 | 2.40631 |
| OH | 167.00 | 168.9 | 166.173 | 1.98 | 1.92973 | 1.92924 |
| (Bottom) | | | | | | |

^{a}NMR data that appears in the scientific literature.
^{b}Experimental data acquired using a 500 MHz NMR.
^{c}o = ortho; m = meta; p = para

In order to better understand the interaction between the copper (II) cation and the medicinal agent taxol, a prototype example of an amine containing medicinal agent, well established computational methods are employed. In addition to demonstrating an increase in water solubility as evidence by an increase in charge, a shift in dipole moment is also shown as the preference of the copper (II) cation for the nitrogen atom.

To enhance and better understand this discovery, a computer based study involving 126 copper(II)-taxol complexes, 126 monohydrated copper-taxol-H₂O complexes, and 2 basic taxol structures were computationally constructed. We evaluated a total of two hundred and fifty four molecules for this analysis. Experimental data indicates the copper (II) ion forms a hexavalent, octahedral geometry. Chelating compounds primarily form bonds with metal atoms by forming M*-O, or M*-N bonds. Copper specifically has a high affinity for amines. Considering these properties, all copper complexes were formed with a Cu—N bond and 5 Cu—O bonds. Given the molecular formula of taxol, (C₄₇H₅₁NO₁₄) and assuming that a Cu—N bond is present in all molecules, a permutations equation can be used to derive the total number of possible copper-taxol complexes (Table 11, 12).

For copper-taxol complexes there are a total of over two-thousand (2002) possible Cu—O and Cu—N bond combinations employing a hexavalent geometry, and for Cu-taxol-H₂O complex there are over one thousand (1001) possible bonding combinations with a hexavalent geometry. These combinations assume that all oxygen atoms in the taxol molecule are available for bonding to the copper (II) ion. In this study, taxol analogues (breaking a bond in the taxol molecule to form a new bond with the cation) are not considered, thus reducing the number of possible oxygen atoms for bonding from 14 to 9. This results in 126 possible copper(II)-taxol complexes, and 126 possible copper-taxol-H₂O complexes. Of the 252 possible combinations (126=126), those that had any Cu—O or Cu—N bond distances greater than 2.9 Å after performed calculations were eliminated as these bonds can be considered to be too long and lack covalence. The long bonds indicate a weak bond and would result in a weak complex, which is likely to dissociate.

The remaining molecules were used to generate tables 13 and 14. Of the 126 possible copper(II)-taxol complexes, four were shown to match the criteria set forth above, and these structures are summarized in table 13 along with the two taxol complexes included in this study. Of the 126 possible copper-taxol-H₂O complexes, 16 were shown to match the criteria set forth above, and they are shown in table 14. With the hexavalent copper, the computational studies indicate zero or one of the six inner sphere sites can be occupied by water while the rest are occupied by a single nitrogen (amine) and oxygen atoms on the taxol structure. Given that experimental results do not show any waters in the inner sphere (one could be loosely bound and lost in the mass spectrometry ionization process), these data are in agreement.

A molecules dipole moment (D, Debye) and molecular volume (V, A³) are two important factors when determining a medicinal agents solubility in different solvents, particularly water. These two parameters form a DN ratio that is important to fully understand or predict solubility. While dipole moment is an important factor for solubility, the volume over which the charge needs to be considered.

Table 12 provides the dipole moment (D), molecular volume (V), and the DN ratio for a number of common solvents for comparison and reference in this study. Calculated variables extend from the non-polar solvent hexane (DN of 0.0) to the polar solvent water (D/V of 0.090) (Table 12). Previous studies developed a parameter called the Aqueous Stability Factor (ASF) to indicate an individual complex's solubility and stability in an aqueous environment. This parameter combines the calculated complex energy (C), average Cu—O+Cu—N bond length (L), dipole moment (D), and molecular charge (Z):

ASF=(E*L)/(D*Z) (8)

The complex stability is approximated by the complex energy, because the smaller or more negative the complex energy, the more stable it should be. Bond distance is a function of covalency, so the Average Bond Length helps us determine how strong the bond is with the chelated atom. Dipole Moment helps us determine solubility to a degree, so a larger Dipole value should signify greater solubility. The Molecular Charge is included because increasing charges also improve molecular solubility. The initial Aqueous Stability Factor value is expressed as units of J*m/D. Molecular Volume has been added to better correlate the ASF with a complex's solubility in solution. The modified version is given as:

ASF=(E*L)/((D/V)*Z) (9)

Which can be rearranged to:

ASF=(E*L*V)/(D*Z) (10)

The improved ASF is used in this study an expressed as units J*m⁴/D.

In Tables 13 and 14, column 1 refers the bonding configuration of the central Cu atom to the respective oxygen atoms. Since one nitrogen atom is located in the taxol structure number labeling is not required. In all complexes, the single Nitrogen occupies the first binding site. In Table 13, there are five numbers under the Configuration column referring to the five Cu—O bonds that occupy binding sites 2-6. In Table 14 there are four numbers referring to the four Cu—O bonds occupying binding sites 2-5, with the sixth binding site being occupied by H₂O. In all complexes Cu is chelated as a central hexavalent atom with an octahedral geometry.

Column 2 lists the Method under which each complex was calculated, where NS=Neutral Singlet, CS=Cation Singlet, and CD=Cation Doublet. Column 3 lists the bond distances used to calculate the Average Bond Length. In Table 13, the values listed 2^{nd} to 6^{th} are in the same order as, and correspond to the configuration provided in Column 1, with the single Cu—N bond listed first. In Table 14, the values listed 2^{nd} to 5^{th} are in the same order as, and correspond to the configuration provided in Column 1, with the first number being the Cu—N bond, and the last the Cu—H₂O. In both charts, Bond Average, Volume, Dipole, Energy, and Charge are the values used to calculate the ASF value present in Column 11. Also provided in the chart are D/V values in Column 8 and Molecular Area in Column 5. Table 15 provides the average values for each group of complexes, uncomplexed taxol, copper-taxol complex, and the copper-taxol-water complex.

TABLE 11
Total possible number of copper(II)-taxol combinations
based on the permutations equation n!/((r!(n − r)!).
Possible combinations n!/((r!(n − r)!)

| Group | N | R | Total |
|---|---|---|---|
| Cu-Taxol | 14 | 5 | 2002 |
| Cu-Taxol-H₂O | 14 | 4 | 1001 |
| Copper(II)-taxol refined | 9 | 5 | 126 |
| Cu-Taxol-H₂O refined | 9 | 4 | 126 |

TABLE 12
A list of common solvents with their calculated dipole moment (D),
molecular volume (Å³), and D/V ratio (Debye/Å³).

| | Dipole | Molecular | D/V |
|---|---|---|---|
| Name | Moment (D) | Volume (Å³) | (Debye/Å³) |
| Water | 1.74 | 19.24 | 0.09 |
| Methanol | 1.54 | 40.66 | 0.038 |
| Ethanol | 0.148 | 59.08 | 0.025 |
| 1-Propanol | 0.159 | 77.37 | 0.02 |
| 1-Butanol | 1.6 | 95.69 | 0.017 |
| 1-3 Butanediol | 3.23 | 102.19 | 0.031 |
| 1-Pentanol | 1.41 | 114.06 | 0.012 |
| 1-Octanol | 1.62 | 168.95 | 0.0096 |
| Hexane | 0 | 124.8 | 0 |

TABLE 13
A summary of taxol and copper(II)-taxol complex computational results (note E represents exponent or to the power of ten).

| | | | | | | Dipole | | | | |
|---|---|---|---|---|---|---|---|---|---|---|
| | | Cu—N, Cu—O | Bond | | Volume | Moment | | | | |
| | | Bond | Average | | (V, | (D, | | Energy | | ASF |
| Configuration | Method | Distances (Å) | (Å) | Area (Å²) | Å³) | Debye) | D/V | (kJ/mol) | Charge | (J * m⁴/D) |
| Taxol | CD | NA | 1 | 793.73 | 827.88 | 5.41 | 0.00653 | 1316.7005 | 1 | 2.01492E−32 |
| Taxol | NS | NA | 1 | 784.63 | 826.94 | 5.03 | 0.00608 | 1316.7005 | 0 | 2.16468E−32 |
| {1, 2, 3, 7, 8} | CS | 1.901, 1.845, | 1.85466 | 778.82 | 830.84 | 1.95 | 0.00234 | 1438.7161 | 1 | 1.1369E−31 |
| | | 1.862, 1.431, | | | | | | | | |
| | | 2.182, 1.907 | | | | | | | | |
| {1, 2, 3, 7, 13} | CS | 1.830, 1.856, | 1.859 | 769.18 | 829.14 | 20.37 | 0.02456 | 1245.8209 | 1 | 9.42696E−33 |
| | | 1.860, 1.871, | | | | | | | | |
| | | 1.874, 1.863 | | | | | | | | |
| {1, 2, 7, 8, 13} | CS | 1.911, 1.861, | 1.8851 | 769.39 | 836.36 | 15.59 | 0.01864 | 1579.0774 | 1 | 1.59698E−32 |
| | | 1.865, 1.941, | | | | | | | | |
| | | 1.877, 1.856 | | | | | | | | |
| {2, 3, 7, 8, 13} | CS | 1.924, 1.853, | 1.997 | 752.61 | 825.43 | 10.43 | 0.01263 | 2470.5863 | 1 | 3.90458E−32 |
| | | 2.307, 2.110, | | | | | | | | |
| | | 1.917, 1.871 | | | | | | | | |

TABLE 14
Cu-Taxol-H₂O Complex Computational Results. The first bond distance value is the Cu—N bond, the subsequent 4 bonds listed
are the Cu—O bonds, and final bond value is the Cu—H₂O bond (note E represents exponent or to the power of ten).

| | | Bond | | | | | | | | |
|---|---|---|---|---|---|---|---|---|---|---|
| | | Distance (Å) | Bond | | | | | | | |
| | | Cu—N/Cu—O/ | Average | Area | Volume | Dipole | D/V | Energy | | ASF |
| Configuration | Method | Cu—H₂O | (Å) | (Å²) | (Å³) | (D) | (Å) | (kJ/mol) | Charge | (J * m⁴/D) |
| {1, 2, 3, 5} | CD | 1.879, 1.860, | 1.868 | 794.64 | 849.06 | 6.83 | 0.0080 | 1270.5832 | 1 | 2.95104E−32 |
| | | 1.855, 1.854, | | | | | | | | |
| | | 1.869, 1.893 | | | | | | | | |
| {1, 2, 3, 7} | CD | 1.865, 1.861, | 1.862 | 789.31 | 846.57 | 18.7 | 0.02208 | 876.4494 | 1 | 7.39E−33 |
| | | 1.850, 1.852, | | | | | | | | |
| | | 1.863, 1.884 | | | | | | | | |
| {1, 2, 3, 8} | CD | 1.879, 1.855, | 1.872 | 787.22 | 848.57 | 7.51 | 0.00885 | 1248.9703 | 1 | 2.64184E−32 |
| | | 1.862, 1.852, | | | | | | | | |
| | | 1.895, 1.889 | | | | | | | | |
| {1, 2, 3, 13} | CD | 1.896, 1.852, | 1.872 | 791.44 | 848.73 | 5.91 | 0.00696 | 1920.5067 | 1 | 5.16348E−32 |
| | | 1.843, 1.862, | | | | | | | | |
| | | 1.888, 1.892 | | | | | | | | |
| {1, 2, 5, 13} | CD | 1.887, 1.927, | 1.949 | 766.59 | 840.64 | 13.16 | 0.01565 | 1228.5981 | 1 | 1.53025E−32 |
| | | 2.249, 1.863, | | | | | | | | |
| | | 1.868, 1.905 | | | | | | | | |
| {1, 2, 7, 8} | CD | 1.890, 1.872, | 1.868 | 794.65 | 846.34 | 17.43 | 0.0205 | 2889.4995 | 1 | 2.62205E−32 |
| | | 1.845, 1.855, | | | | | | | | |
| | | 1.858, 1.893 | | | | | | | | |
| {1, 2, 7, 13} | CD | 1.846, 1.864, | 1.892 | 786.91 | 845.66 | 14 | 0.01655 | 3196.9308 | 1 | 3.65361E−32 |
| | | 1.855, 1.956, | | | | | | | | |
| | | 1.880, 1.951 | | | | | | | | |
| {1, 3, 5, 13} | CD | 1.904, 1.846, | 1.889 | 774.14 | 846.12 | 9.17 | 0.01083 | 1389.983 | 1 | 2.42358E−32 |
| | | 1.946, 1.854, | | | | | | | | |
| | | 1.892, 1.896 | | | | | | | | |
| {1, 3, 7, 8} | CD | 1.903, 1.861, | 1.877 | 789.45 | 848.89 | 14.63 | 0.01723 | 3053.5593 | 1 | 3.32566E−32 |
| | | 1.860, 1.864, | | | | | | | | |
| | | 1.863, 1.911 | | | | | | | | |
| {1, 3, 7, 13} | CD | 1.891, 1.856, | 1.870 | 783.97 | 846.87 | 12.38 | 0.01461 | 1526.7282 | 1 | 1.95316E−32 |
| | | 1.849, 1.865, | | | | | | | | |
| | | 1.873, 1.887 | | | | | | | | |
| {1, 7, 8, 13} | CD | 1.891, 1.857, | 1.878 | 788.43 | 850.47 | 16.85 | 0.01981 | 1398.5588 | 1 | 1.32591E−32 |
| | | 1.877, 1.866, | | | | | | | | |
| | | 1.881, 1.898 | | | | | | | | |
| {1, 9, 11, 13} | CD | 1.940, 1.845, | 1.888 | 760.72 | 840.01 | 9.22 | 0.0109 | 2447.6548 | 1 | 4.21209E−32 |
| | | 1.872, 1.874, | | | | | | | | |
| | | 1.898, 1.904 | | | | | | | | |
| {2, 3, 7, 8} | CD | 1.907, 1.914, | 1.938 | 787.24 | 843.15 | 13.09 | 0.01552 | 1886.933 | 1 | 2.35566E−32 |
| | | 1.827, 1.929, | | | | | | | | |
| | | 2.165, 1.887 | | | | | | | | |
| {2, 3, 7, 13} | CD | 1.963, 1.858, | 1.901 | 790.08 | 844.45 | 4.44 | 0.0052 | 1974.5689 | 1 | 7.13976E−32 |
| | | 1.864, 1.946, | | | | | | | | |
| | | 1.873, 1.903 | | | | | | | | |
| {2, 7, 8, 13} | CD | 1.877, 1.847, | 1.939 | 784.87 | 844.96 | 16.24 | 0.01921 | 2226.6473 | 1 | 2.24713E−32 |
| | | 1.843, 2.058, | | | | | | | | |
| | | 2.104, 1.909 | | | | | | | | |
| {3, 7, 8, 13} | CD | 1.909, 1.865, | 1.964 | 750.26 | 845.03 | 11.51 | 0.01362 | 2493.3358 | 1 | 3.59547E−32 |
| | | 1.911, 2.251, | | | | | | | | |
| | | 1.879, 1.970 | | | | | | | | |

TABLE 15
Average Values of complexes grouped by type for comparison (note E represents exponent or to the power of ten).

| | Bond | | | | | | | | |
|---|---|---|---|---|---|---|---|---|---|
| | Average | | Volume | Dipole | | Energy | | ASF | |
| | (Å) | Area (Å²) | (Å³) | (Debye) | D/V | (kJ/mol) | Charge | (J * m⁴/D) | V/A (Å) |
| Taxol | N/A | 789.18 | 827.41 | 5.22 | 0.00630 | 1316.7005 | 0 | — | 1.04847 |
| Cu-Taxol | 1.898958 | 767.5 | 830.4425 | 12.085 | 0.01454 | 1683.55 | 1 | 4.45332E−32 | 1.08213 |
| Cu-Taxol- | 1.895802 | 782.495 | 845.97 | 11.9418 | 0.01411 | 1939.34 | 1 | 2.99248E−32 | 1.08136 |
| H₂O | | | | | | | | | |

Comparing the values present in table 14 and 15, it can be observed that there is only a negligible difference in the average bond distances between both the copper(II)-taxol and the copper(II)-taxol-H₂O complexes. The average volume of the copper(II)-taxol complexes is very similar to that of the uncomplexed taxol molecule. The average volume of the copper(II)-taxol complexes is 830.442 Å³ and the volume of uncomplexed taxol is 827.41 Å³, showing an average difference of 0.367%. The average difference is monohydrated complexes is 2.24%. The dipole moment values rose drastically for the copper(II)-taxol (0.0141D) and copper(II)-taxol-H₂O (0.0141D) complexes verses uncomplexed taxol molecule (0.0063D), demonstrating that that solubility is improved in an aqueous environment for the copper(II)-taxol complexes. Taxol is often measured as a sodium adduct in mass spectrometry studies but in water this is a strong electrolyte (Na-taxol [symbol] Na⁺+taxol) so the +1 charge associated with the sodium ion does not apply and the ASF for uncomplexed can not be calculated.

The D/V ratios also rose significantly for each group of complexes as well in relation to basic taxol. The average energy of both groups of complexes also rose in relation to the uncomplexed taxol. These computational exercises demonstrate that the copper (II)-taxol complex has a significantly higher water solubility, important for the physiological environment.

The binding of copper (II) to quinine and taxol is demonstrated. The World Health Organization of essential medicines includes many pharmaceutical agents that contain amines and have low water solubility. We have also used computational methods to show the water solubility of many of these species can be improved by binding the copper (II) ion. The copper (II) ion presents an economical method to increase the medical efficiency of hundreds of pharmaceutical agents currently on the market.

What is claimed is:

1. A method of utilizing specific copper ions as complexing binding agents for nitrogen containing drugs in the treatment of diseases.
(a) the copper (I) cation binds to the medicinal agent by at least one nitrogen atom.
(b) the copper (II) cation binds to the medicinal agent by at least one nitrogen atom.
(c) the copper ions may switch oxidation states from doubly charged cation to singly cation to neutral and vice versa, while bound to the medicinal agent.
(d) the copper cation is serving as a delivery agent to enhance the efficacy of the pharmaceutical agent.

2. A method according to claim 1 whereby the intracellular components being targeted by the carrier copper ion includes, microsomal material, mitochondrial material, ribosomal material, nuclear material, the cytoskeleton, and/or other cytoplasmic components.

3. A method for enhancing the efficacy of a pharmacologically active nitrogen containing agent administering the active agent to a region of a patient's body in combination with a copper atom.

4. The method of claim 3, where the mole to mole ratio of copper ion to medicinal agent, respectfully, is between 0.5/1.0 to 100.0/1.0.

5. The method of claim 3, wherein the formulation is administrated as a solid, liquid, mist or cream.

6. The method of claim 3, wherein the formulation is administrated as a tablet, an IV, an aqueous injection, a nonaqueous injection, a paste, a gel, a lotion, a transdermal or some other accepted method of drug delivery.

7. The method of claim 1, wherein the medicinally active agent contains a nitrogen atom.

8. The method of claim 1, wherein the active agent contains a primary amine.

9. The method of claim 1, wherein the active agent contains a primary amine.

10. The method of claim 8, wherein the active agent contains a secondary amine.

11. The method of claim 8, wherein the active agent contains a tertiary amine.

12. The method of claim 1, wherein the active agent contains a nitrogen-containing heterocycle.

13. The method of claim 13, wherein the heterocycle is non-aromatic.

14. The method of claim 14, wherein the heterocycle is aromatic.

15. The method of claim 1, wherein the active agent contains an azo group.

16. The method of claim 1, wherein the active agent contains an amine group.

17. The method of claim 1, wherein the active agent may contain one or more of any nitrogen containing functional group including the amines, azides, azines, azo, carbamate, cyanate, diazo, diazonium, enamine, hydrazine, hydrazone, hydroxamic acid, hydroxylamine, imide, imine, nitrate, nitrile, nitrite, nitro, nitrosamine, nitroso, nitroso, oxime, sulfonamide, sulfinylimine, sulfonamide, sulfonylimine, N-oxide, azoxy, carbodiimide, cyanamide, dithiocarbamate, guanidine, isonitrile, nitrone, nitronate, phosphoramidite, phosphoramidate, semicarbazide, semicarbazone, sulfoximine, thioamide, ammonium and or ammonia.

18. The method of claim 18 where a copper ion may bind a least one functional group found in the medicinally active ingredient.

19. The method of claim 18 where one copper ion may bind more than one nitrogen containing compound forming either a metal ligand complex where MLₓ(x>1.0).

20. The method of claim 18 where one copper ion may bind more than one nitrogen containing compound forming an aggregate where the copper ion may initiate the aggregation process but may not directly bind all molecules involved in the aggregate.
